(19) United States
(12) Patent Application Publication
(10) Pub. No.: US 2002/0064876 A1
(43) Pub. Date: May 30, 2002

(54) NOVEL GENE THERAPY METHODS FOR THE TREATMENT OF SKIN DISORDERS

(76) Inventor: KYONGGEUN YOON, BERWYN, PA (US)

Correspondence Address: WILLIAM J MCNICHOL ESQ, REED SMITH SHAW & MCCLAY LLP, 2500 ONE LIBERTY PLACE, 1650 MARKET STREET, PHILADELPHIA, PA, 19103-7301, US

(21) Appl. No.: 09/473,872

(22) Filed: Dec. 28, 1999

(51) Int. Cl. A61K 48/00; A01K 67/027; C07H 21/04

(52) U.S. Cl. 435/440; 514/44; 435/455; 435/463; 536/23.1; 800/9; 800/14

(57) ABSTRACT

This invention provides methods for modifying a selected gene in cells of a mammalian skin at one or more locations by delivering to the skin cells an effective amount of a composition having a chimeric RNA-DNA oligonucleotide for causing heritable modifications in the selected gene so that the heritable modifications result in phenotypic changes at the locations of the mammalian skin. The invention specifically provides a method for permanent gene correction of a gene mutation by an RNA-DNA oligonucleotide (RDO) in vivo. By this method, a point mutation in the albino BALB/c mouse tyrosinase gene in vivo has been corrected thereby providing for permanent and inheritable restoration of tyrosinase enzymatic activity, melanin synthesis, and pigmentation changes in melanocytes of skin at the treated locations. Both topical application and intradermal injection of this oligonucleotide to mice skin resulted in dark pigmentation of several hairs in localized area.

Pursuant to 35 U.S.C. §202(c) it is acknowledged that the U.S. Government may have certain rights in invention described herein, which was made in part with funds from the National Institute of Health, Grant Number AR44350 and AR38923.

### FIELD OF THE INVENTION

The present invention relates to method of treating skin disorders in vivo by correcting point and frameshift mutations in genes or by creating point and frameshift mutations in genes that cause certain phenotypic changes. More in particular the invention relates to methods of treating genetic skin disorders by generating in situ genetic alterations into genes related to these disorders and acquired skin diseases by inactivtion or modification of cytokine genes and cell adhesion genes.

### BACKGROUND OF THE INVENTION

There are a number of dermatological disorders which are of a major concern to humans. These include genetic skin disorders such as epidermal fragility (epidermolysis bullosa, EB) disorders, keratinization disorders, hair disorders, pigmentation disorders, porphyrias, skin cancer and multisystem disorders and acquired skin disorders such as psoriasis. Psoriasis is common, chronic disease, affecting approximately 2% of the population in western countries. Psoriasis represents an inflammatory skin disorder characterized by loss of normal cellular homeostasis, resulting in epidermal hyperproliferation, defective differentiation and inflammation. It has been reported that psoriasis originates from T-cell activation and increased sensitivity of psoriatic keratinocytes to T-cell derived cytokines.

The current treatment for patients suffering from genetic skin disorders is primarily symptomatic reduction of skin trauma, and treatment of bacterial infection with no cure. Most of these disorders have been genetically defined and were shown to be the result of mutations in the genes controlling normal phenotypes. For example, it is well known that the group of inherited blistering skin diseases, collectively known as epidermolysis bullosa (EB), are manifested due to mutations in the genes expressed in the cutaneous basement membrane zone primarily by basal keratinocytes. By simply correcting the genes carrying the mutations may offer an effective solution. Similarly, acquired skin diseases such as psoriasis can be corrected by creating mutations, for example, in the genes controlling T-cell activation. Thus, this information can be utilized in developing gene therapy strategies to treat the patients.

Current technologies for cutaneous gene therapy are based on two approaches; direct in vivo and ex vivo methods. Direct in vivo methods administer genetic material directly to the skin by injection, topical application, or particle bombardment (Khavari et al., 1997, Adv. Clin. Res., 15(1):27). The ex vivo approach involves removal of tissue from patients for culture, gene transfer to the cultured cells, followed by grafting the recombinant cells to the patient (Greenhalgh et al., 1994, J. Invest Derm., 103, 63S-69S).

Retroviral mediated gene transfer of the growth hormone and factor IX into proliferating keratinocytes has been shown to result in secretion of these proteins in vitro. When these cells were grafted to the mice, both proteins have been detected in mouse blood stream. These observations have attested to the possibility that epidermis can be engineered to be a bioreactor, geared to the secretion of gene products that have a local or systemic effect. Adenovirus mediated gene transfer of the reporter plasmid β-galactosidase and the human α1-antitrypsin was carried out by transplantation of the adenovirus-infected keratinocytes to syngeneic mice. A substantial expression of the α1-antitrypsin was detected over 14 days in the bloodstream of the mice.

Although the viral gene delivery has made an impressive advancement toward clinical applications, several drawbacks exist. For example, retroviral transfer requires the cell division for infection, thereby limiting infections of the slowly cycling stem cells. It has also been difficult to generate a stable and a high titer of virus effective for infection. Moreover, several studies have documented the gradual inactivation of expression from viral promoters, even though the vector DNA clearly remains present. Also, there is a safety concern of a possible generation of replication competent retrovirus. Adenoviral delivery system has the advantage in that it does not require the target cell to be mitotically active for infection, and a high titer of the recombinant virus can be generated. By the same token, adenoviral vectors are likely to infect non-replicating, terminally differentiated cells, thus limiting expression to the transit times of these cells. Moreover, infected cells have been a target of immune system and were shown to be actively eliminated from the body. Current viral delivery system has a limitation in the size of the genes that can be packaged into a viral particle efficiently. Further for example, several genes involved in the pathoetiology of different forms of EB, such as the genes encoding type VII collagen, the 230-kD and the 180-kD bullous pemphigoid antigens, the β4 integrin and the subunit polypeptides of laminin 5 are relatively large, ranging up to 50 kb for the genomic sequence and 10 kb for the cDNA. Consequently, it is difficult to deliver the entire gene or a full-length cDNA by currently available retroviral or adenoviral vectors. The plasmid-liposome complexes have advantages as gene transfer vector since the size of the delivered gene is unlimited. However, these modes of gene delivery, virus or liposome, result in a random integration of the DNA.

An ideal situation would be to return the cloned gene into its homologous location in the genome at the target site. In the past, this approach has not been practical since homologous recombination is an extremely rare event in mammalian cells. Recently, a novel gene therapy strategy has been developed that involves the use of a unique chimeric oligonucleotide containing both RNA and DNA. Genetic-based strategies capable of correcting mutations have been developed by using a chimeric RNA-DNA oligonucleotide (RDO) (Yoon, et al., 1996, Proc Nad Acad Sci USA, 93: 2071-2076; Cole-Strauss, et al., 1996, Science, 273: 13 86-13 89; Xiang, et al., 1997, J Mol Med, 75: 829-835; Kren, et al., 1997, Hepatology, 25: 1462-1468; Kren, et al., 1998, Nat Med, 4: 285-290; Alexeev, et al., 1998. Nat Biotech, 16. 1443-1346; Santana, et al., 1998. J Invest Dermatol, 111:1172-1177; Yoon, K. 1999, Biogenic Amines, 15: 137-167). This approach has the potential to correct the desired mutation while maintaining a complex genomic organization important for the appropriate expression and regulation of genes. The feasibility of this method was demonstrated first by episomal correction of a point mutation (Yoon, et al., 1996, Proc Nad Acad Sci USA, 93: 2071-2076).

Additionally, animal models facilitate disease pathology analysis and provide a system for new therapeutics exploration. The potential now exists in experimental systems to generate animal models of EB. Transgenic mice manifesting a striking resemblance to that of a heterogeneous group of autosomal dominant simplex EB were generated by expression of truncated keratin 14 proteins. An alternative method to generate animal model is gene-targeting in embryonic stem (ES) cells. Target gene inactivation was achieved by selection of cells in which a rare homologous recombination occurred between the introduced DNA and target gene. However, current animal models generated either by transgenic or embryonic stem cell technology result in a large alteration of the mouse genome. Although these models proved useful for identification of certain human genetic disease etiology, they do not reproduce the mutations present in human genetic diseases. Thus, an ideal situation would be to generate a specific mutation in animals which reproduces or mimics those present in human patients.

In the present invention, a method for localized in vivo genotypic and phenotypic modifications in skin by using RNA-DNA oligonulecotides has been demonstrated. The method enabled correction of a mutant base in chromosomal targets in a sequence-specific and inheritable manner. More, specifically a high frequency of gene correction in vivo has been demonstrated. It has also been shown by the present invention that an animal model can be created carrying dominant mutation in a wild type EB gene that results in phenotyic alteration.

### SUMMARY OF THE INVENTION

The present invention relates to methods for treating skin disorders in vivo. The skin disorders that can be treated by the methods of the present invention can be genetic or acquired skin disorders. The skin disorder is remedied by modifying a gene which modification is done by correcting point and frameshift mutations in genes or by creating point and frameshift mutations in genes that cause certain phenotypic changes. More in particular the invention relates to methods of treating genetic skin disorders by generating in situ genetic alterations into genes related to these disorders and acquired skin diseases by inactivtion or modification of cytokine genes and cell adhesion genes.

The skin gene can be tyrosinase (Tyr), COL7A1, LAMA3, LAMB3, LAMC2, COL17A1, ITGA6, ITGB4, PLEC1, KRT5, KRT14, PKP1, KRTI, KRT10, KRT9, KRT16, LOR, KRT2e, KRT6a, KRT 16, KRT 17, STS, TGM1, GJB2, GJB3, ATP2A2, DSP, DSG1, HR, hHB1, hHB6, PAX3, TYR, TYRP-1, OCA2, OA1, MITF, HPS, FECH, UROS, URO-D, XPA, XPB, XPC, XPD, XPG, CSB, PTC, STK11/LKB1, PTEN, PTEN, XPB, XPD, WHN, GLA, ATM, ENG, ALK-1, or PPO gene.

According to one aspect of the invention, a method of modifying a selected gene that is naturally expressed in cells of the human skin is provided. This method includes delivering to cells of a human skin at one or more locations cells an effective amount of a composition which includes a chimeric RNA-DNA oligonucleotide and a pharmaceutically acceptable carrier such that the stable genetic modifications are made to the selected gene which result in phenotypic changes at these locations of the human skin. In one emodiment, the stable genetic modification is in an epidermal fragility disorder gene. In another embodiment the stable genetic modification is in a keratinization disorder gene.

The basic design of the chimeric RNA-DNA oligonucleotide includes (a) a first string of nucleotides wherein the first string is made of at least four contiguous deoxyribonucleotides flanked on each side by at least nine ribonucleotides; and (b) a second string of nucleotides that is fully complementary to the first string of nucleotides or is fully complementary to the first string of nucleotides except that the first and second strings have one mismatched base pair in the region corresponding to the deoxyribonucleotides of the first string, wherein the second string has the same number of deoxyribonucleotides as in the first string of nucleotides, and wherein one or more nucleotides of the chimeric RNA-DNA oligonucleotide are nuclease protected, and wherein the chimeric RNA-DNA oligonucleotide has nucleotides in the first and second strings that are fully complementary to a segment of DNA of the selected gene except that the first string has one mismatching deoxyribonucleotide that defines the site of modification in the selected gene.

A modified design of the chimeric RNA-DNA oligonucleotide includes (a) a first string of nucleotides wherein the first string is made of at least 20 ribonucleotides; and (b) a second string of deoxyribonucleotides having the same number of deoxyribonucleotides as in the first string of nucleotides, wherein the second string is fully complementary to the first string of nucleotides except that the second string has a deoxyribonucleotide that forms a mismatched base pair with the corresponding nucleotide in the first string, and wherein one or more nucleotides of the chimeric RNA-DNA oligonucleotide are nuclease protected, and wherein the chimeric RNA-DNA oligonucleotide has nucleotides in the first and second strings that are fully complementary to a segment of the two strands of DNA of the selected gene except that the deoxyribonucleotide in the second string also forms a mismatched base pair with the corresponding deoxyribonucleotide in the DNA strand of the selected gene which mismatched base pair defines the site of modification in the selected gene.

Another variation of the basic the chimeric RNA-DNA oligonucleotide includes (a) a first string of nucleotides wherein the first string is made of at least four contiguous deoxyribonucleotides flanked on each side by at least nine ribonucleotides; and (b) a second string of nucleotides that is fully complementary to the first string of nucleotides or is fully complementary to the first string of nucleotides except that the first and second strings have one mismatched base pair in the region corresponding to the deoxyribonucleotides of the first string, wherein the second string has the same number of deoxyribonucleotides as in the first string of nucleotides, and wherein one or more nucleotides of the chimeric RNA-DNA oligonucleotide are nuclease protected, and wherein the chimeric RNA-DNA oligonucleotide has nucleotides in the first and second strings that are fully complementary to a segment of DNA of the selected gene except that the first and second strings have one, two or four pairs of nucleotide insertions or deletions that defines the site of modification in the selected gene.

The stable genetic modification is correction of a mutation or generation of a mutation, where the mutation is a point mutation or a frame shift mutation. In some emobodiments the mutation is a dominant mutation. The phenotypic changes include the correction of a skin disorder such as the correction of albinism, an epidermal fragility disorder or a keratinization disorder.

According to another aspect of the invention, A method of modifying a selected gene in cells of an animal skin at one or more locations is provided which includes delivering to the cells an effective amount of a composition comprising a chimeric RNA-DNA oligonucleotide and a pharmaceutically acceptable carrier such that the stable genetic modifications are made to the selected gene which result in phenotypic changes at said locations of the animal skin, wherein the animal is selected from the group consisting of a mouse, a rabbit, a goat, a monkey, a pig and a cow.

According to still another aspect of the invention, an animal model having a skin disorder at one or more locations of its skin is provided where the skin disorder is due to the treatment at these locations with a composition having a chimeric RNA-DNA oligonucleotide targeted to a selected skin gene. The skin disorder can epidermal fragility disorder, a keratinization disorder or albinism disorder.

According to yet another aspect of the invention, a method of correcting a mutation in a tyrosinase gene in cells of a mammalian skin at one or more locations is provided. This method includes the step of delivering to the skin cells an effective amount of a composition comprising a Tyr-A RNA-DNA oligonucleotide for causing stable genetic correction in the tyrosinase gene and a pharmaceutically acceptable carrier such that the correction results in restoration of tyrosinase enzyme activity at these locations of the mammalian skin. The skin of the mammalian animal is selected from any of the following: a human, a mouse, a rabbit, a goat, a monkey, a pig and a cow skin.

### BRIEF DESCRIPTION OF THE FIGURES

FIG. 1 schematically shows sequences of the Tyr-A RDO and Tyr-B RDO, and the targeted sequences in the tyrosinase gene in one embodiment of the invention.

FIG. 2 illustrates deliveiry of the RDO in the mouse skin in which a fluorescein-conjugated RDO was applied topically (A) and injected intradermally (B) into skin.

FIG. 3 is a composite photograph showing shows Localization of dark hair pigmentation in BALB/c mouse in one embodiment of the invention.

FIG. 4 is to show detection of tyrosinase activity and melanin in skin sections of mice after intradermal injection of the Tyr-A and Tyr-B chimeric oligonucleotide.

FIG. 5 shows RFLP and DNA sequencing analysis of genomic DNA isolated from skin biopsies positive for DOPA staining and melanin synthesis.

FIG. 6 is a photograph of a mouse carrying a genotypic change in COL7A1 gene leading to blister formation.

FIG. 7 is a photograph of a mouse carrying a genotypic change in KRTI 7 gene leading to blister formation.

### DETAILED DESCRIPTION OF THE INVENTION

There are several attractive reasons for considering the skin as a target tissue for gene therapy. First is the accessibility of the tissue which makes it possible to administer therapeutics, to monitor the treatment site and to remove the modified area, if necessary. Human keratinocytes have been cultured successfully in vitro, have produced an intact tissue suitable for grafting, and have been capable of mimicking terminal differentiation and many of the biochemical and genetic properties of intact epidermis when placed in organ culture systems. The epidermis is continuously renewed throughout the adult life span by proliferation of stem cells. The long-term success of cultured epithelial autografts in treating burn patients suggests that epidermal stem cells are present in culture. Thus, stem cell therapy can be applied. In addition, regulatory sequences have been identified for the proliferation and differentiation specific genes of epidermis. These sequences have been employed to express an exogenous genes in the epidermis of transgenic mice and are readily adaptable for gene therapy application for epidermis-specific expression.

There are a number of skin disorders, both genetic and acquired skin disorders which can be treated by applying the methods of the present invention. The present invention can also be used to create animal models that parallel or mimic the course of skin diseases in humans in order to understand human skin diseases and to generate systems to test new drugs and treatments. The genetic skin diseases or disorders with known gene defects are listed in Table 1. A large number of genes involved in different skin disorders were shown to be point mutations or frameshift mutations.

It is well known that mutations involving a change in a single base pair, often called a point mutation, or a deletion of a few base pairs, called a frameshift mutation, generally affect the function of a single gene. Changes in a single base pair may produce (1) a missense mutation, which results in a protein in which one amino acid is substituted for another; or (2) a nonsense mutation, where a nucleotide base change leads to the formation of stop codon. In addition, changes in one or more base pairs (that is not a multiple of three) of nucleotides may produce frameshift mutations which leads to introduction of unrelated amino acids and stop condons.

The present invention can be used to treat skin disorders by correcting or creating point mutations and frameshift mutations in the specific genes related to the disorders.

In one embodiment of the invention, an in vivo genotypic and phenotypic correction of the albino mutation (tyrosinase gene mutation) in skin by RNA-DNA oligonucleotide (RDO) is explained.

Tyrosinase is a key enzyme of melanin synthesis, produced exclusively by melanocytes and sufficient for pigmentation in vitro and in vivo (Halaban, et al., 1993, J Invest Dermatol, 100: 176S-I 85S; del Marmol, et al., 1996, FEBS Lett, 381: 165-168; Shibahara, et al., 1990, J Biochem 189: 455-461; Beermann, et al., 1990, EMBO J, 9: 2819-2826; Tanaka, et al., 1990, Development, 108: 223-227; Huszar, et al., 1991, Development, 113: 653-660; Jackson, et al., 1990, Proc Natl Acad Sci USA, 87: 7010-7014). Melanocytes derived from albino mice contain a homozygous point mutation (TGT→TCT) in the tyrosinase gene resulting in an amino acid sequence change from Cys to Ser at amino acid 85 of the mature tyrosinase (Shibahara, et al., 1990, J Biochem 189: 455-461). This cystein-rich region has been highly conserved among different species and has been suggested to be a heme- or porphyrin-binding domain, which is important for the catalytic activity of tyrosinase (Halaban, et al., 1993, J Invest Dermatol, 100: 176S-I 85S). This single amino acid change was shown to be responsible for the complete inactivation of tyrosinase and an absence of pigmentation (Halaban, et al., 1993, J Invest Dermatol, 100. 176S-I 85S; del Marmol, et al., 1996, FEBS Lett, 381: 165-168; Shibahara, et al., 1990, J Biochem 189: 455-461). However, albino melanocytes form abundant unpigmented premelanosomes as shown by transmission electron microscopy (Huszar, et al., 1991, Development, 113: 653-660; Jackson, et al., 1990, Proc Natl Acad Sci USA, 87: 7010-7014; Bennet, et al., 1989, Development, 105: 379-385). Thus, albino melanocytes contain all necessary cellular components for synthesis and secretion of melanin but lack an active tyrosinase enzyme. Therefore, correction of the point mutation in the tyrosinase gene would result in the restoration of the intracellular enzyme activity followed by pigmentation in vitro and in vivo (Jackson, et al., 1990, Proc Natl Acad Sci USA, 87: 7010-7014).

Mouse hair color is determined primarily by melanocytes, which synthesize and transport melanin to medullary and cortical cells of hair (Straile W. E. 1964, Dev Biol 10: 45-70; Dry. F. W. 1926, JGenet 16: 287-340). Mouse melanocytes are interspersed among matrix keratinocytes in hair follicles. Melanocytes distribute melanin to the surrounding keratinocytes, and as the keratinocytes; move upward, differentiate and are incorporated into the hair shaft, they carry the melanin with them. The hair shaft therefore serves as a record of melanocyte activity and melanin production. The hair cycle is characterized by periods of hair fiber production and proliferation of cells (anagen), a brief regression phase resulting in loss of up to 70% of the hair follicle (catagen) and the resting period of minimal activity (telogen) (Paus, et al., New Eng J Med, in press; Cotsarelis et al., 1990, Cell, 61: 1329-1337). In mouse trunk skin, the majority of melanocytes reside in the hair follicles (Silvers W. K. 1979, Springer-Verlag, New York) and active melanogenisis occurs only during the anagen stage of the hair cycle, indicating a tight coupling between melanin synthesis and the hair cycle (Slominski, et al., 1993, J Invest Dermatol, 100: 816-822).

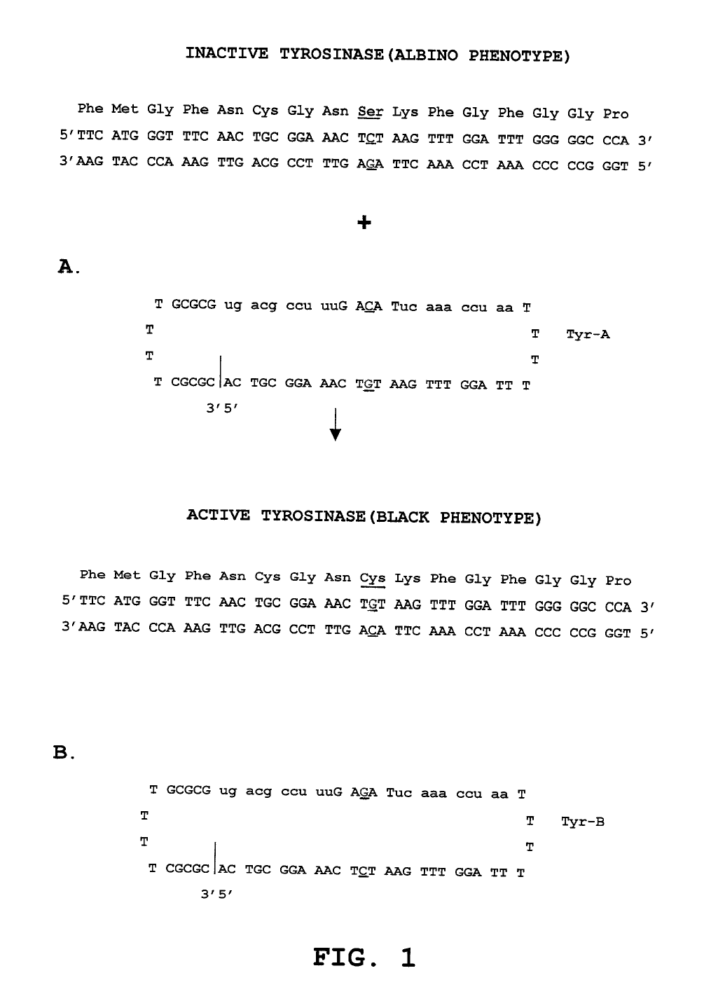

The Tyr-A RDO or the Tyr-A chimeric oligonucleotide was designed to introduce a single base pair substitution TCT→TGT in tyrosinase gene in order to convert an unpigmented albino melanocytes to black-pigmented cells by introducing a single amino acid change, Ser→Cys at amino acid 85 of the mature tyrosinase Tyr-B, which contains an identical sequence to the mutant tyrosinases was used as a control (see FIG. 1).

The Tyr-A shown in FIG. 1 RDO corrected a point mutation in the tyrosinase gene mutation, resulting in the restoration of tyrosinase activity, melanin synthesis and pigmentation changes in a permanent and inheritable manner in cultured melanocytes derived from albino mouse.

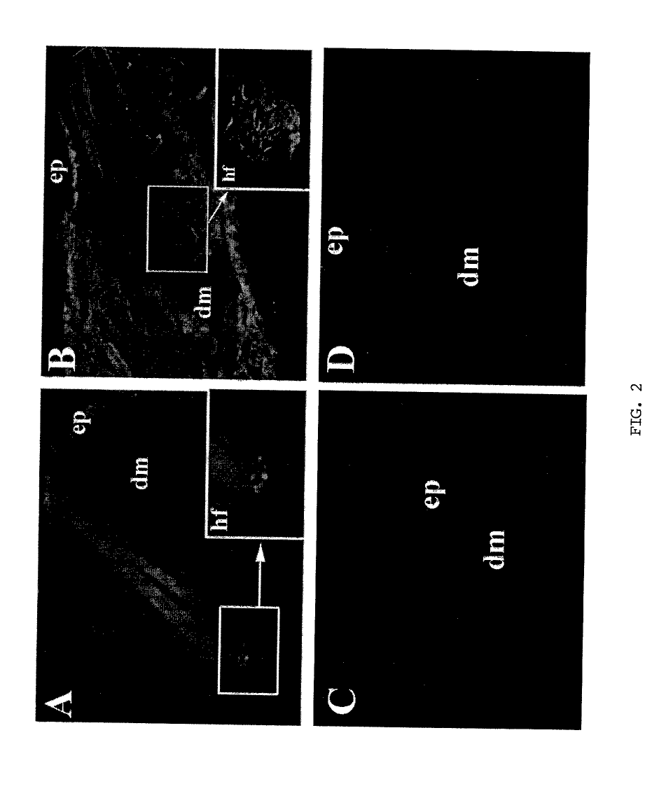

The in vivo application of the Tyr-A RDO and Tyr-B RDO to mouse skin was carried out by two different methods, intradermal injection and topical application. Efficiency of delivery was monitored by a fluorescein-conjugated RDO (RDO-FITC) using fluorescence microscopy as will be described elsewhere in this document. The RDO-FITC was incubated either with Cytofectin™ or SuperFect™. The complex was topically applied or intradermally injected into mouse skin and biopsy was taken 6-8 h after application. The RDO-FITC and Cytofectin™ complex was found to be most efficient in delivery of RDO into hair follicles, where melanocytes reside. The hair follicle is shown as a tubular invagination of the epidermis that encloses a small spike of dermis in its base (FIGS. 2A and 2B). The fluorescence of RDO-FITC was detected in epidermis and hair follicles upon topical application (see FIG. 2A). Intradermal injection resulted in a delivery of RDO in dermis and hair follicles (see FIG. 2B). Both delivery methods were used in further experiment.

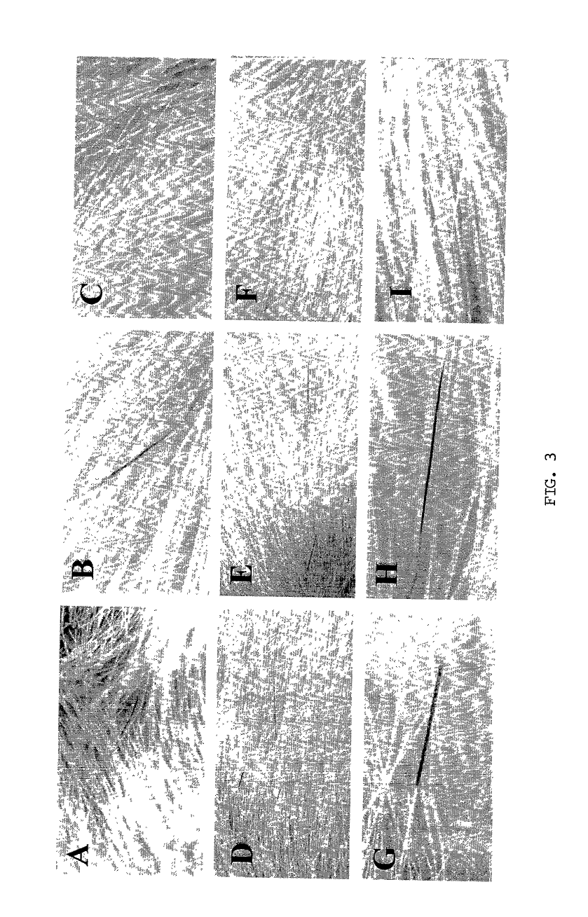

Taking into consideration the synchronized growth and pigmentation of mouse hair, application of RDO/Cytofectin complex was carried out at the beginning of the anagen stage of the mouse hair cycle. The reason for this is that at anagen onset, melanocytes and melanocyte precursors would be proliferating and differentiating to repopulate the new lower follicle at this time. The RDO/Cytofectin complex was either topically applied or intradernally injected into mouse skin. Topical application of liposomes has been used to deliver plasmid DNA to mouse hair follicles in vivo (Li L, et al., 1995, Nat Med, 1: 705-706). The RDO/Cytofectin mixture was applied to the back skin of four 52 day old BALB/c mice every day for 3 days, beginning 2 days after chemical depilation. Following depilation, the follicle enters anagen and produces a new hair follicle and hair (Wilson, et al., 1994, Differentiation, 55:127-136). Several pigmented hairs in the treated areas were detected under the dissecting microscope, 21 days after depilation (FIGS. 3A and 3B). No pigmented hairs were detected in untreated skin or skin treated with the control Tyr-B chimeric digonucleotide (FIG. 3C). Intradermal injection was carried out in two groups of mice. In the first group, four mice were intradermally injected with the RDO/Cytofectin complex (Tyr-A-cytofectin as test treatment with Tyr-B-cytofectin as a control) on the 2^{nd}, 5^{th} and 32^{nd} day after birth. Two days prior to injection, the 30 day old mice were depilated. The mouse hair coat was periodically examined for pigmentary changes. Dark pigmentation of several hairs was observed at 65 to 70 day after birth (FIGS. 3D and E) when the mice were sacrificed. No such hairs were present in untreated skin or in skin treated with the control Tyr-B RDO in both topical application and, intradermal injection (FIGS. 3F and I). Banded pattern of pigmentation in the hair shaft (FIGS. 3G and H) is similar to that of the normal hair (Silvers W. K. 1979, Springer-Verlag, New York). Discontinuous pigmentation of a single hair shaft may be caused by the wild type agouti allele (A) of the BALB/c mouse strain (Halaban, et al., 1993, J Invest Dermatol, 100: 176S-I 85S). The agouti allele induces deposition of black eumelanin, and yellow phaeomelanin during hair cycle, generating an alternating black and yellow hair pigmentation in a single hair (Halaban, et al., 1993, J Invest Dermatol, 100: 176S-I 85S; Huszar, et al., 1991, Development, 113: 653-660). It is also possible that only a subset of melanocytes is corrected by RDO, resulting in limited tyrosinase expression, melanin synthesis, and alternating pigmentation.

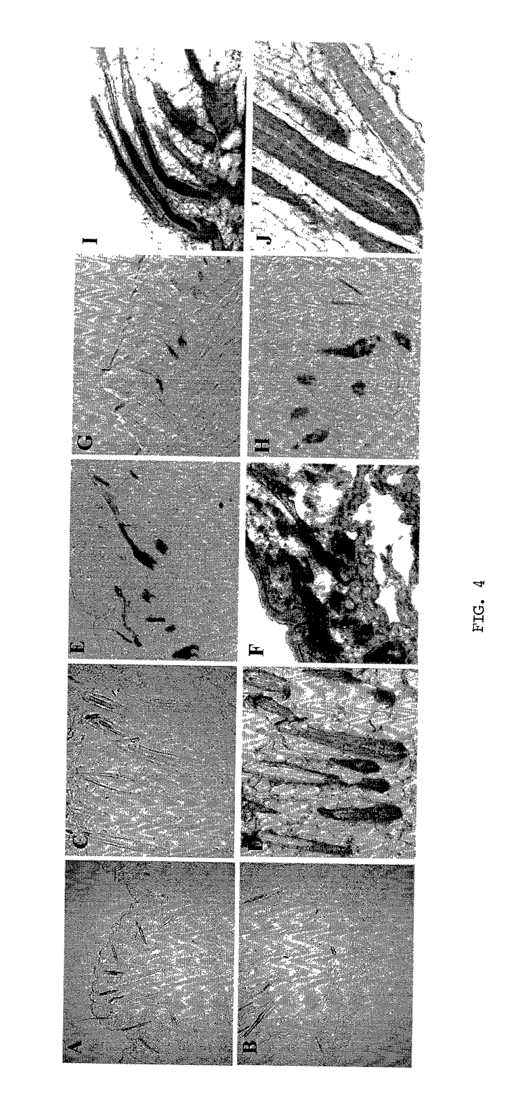

In the second group, seven mice were injected with the RDO/Cytofectin complex on the 2nd, 5th, 32^{nd}, and 67^{th} days after birth. Two days prior to injection, the 30 and 65 day old mice were depilated to induce anagen. Animals were sacrificed at 5 month after birth and skin biopsies were taken from both treated and untreated areas. Tyrosinase activity was detected by oxidation of DOPA to melanin. The DOPA positive hair follicles were observed in two different mice treated with the Tyr-A RDO (FIGS. 4E, G and H). In contrast, none of the anagen hair follicles were pigmented after DOPA reaction in untreated areas (FIGS. 4A, B and C). Active expression of tyrosinase was shown in anagen hair follicles. As expected, no melanocytes in the telogen stage of the hair cycle were positive by DOPA staining. Melanin was also detected using the Fontana-Masson stain (Laws, et al., 1998, Dermatol Surg, 24: 633-636) on paraffin embedded tissue sections (FIG. 41). The number of DOPA positive hair follicles appears to be higher than that of dark pigmented hairs (FIGS. 3 and 4). The pigmented hair and DOPA staining were specific for Tyr-A-treated skin, as indicated by the fact that no change was observed in the Tyr-B-treated areas from the group of control animals. Thus, the localized pigmentation change in 20-30 hairs per 25 mm² was caused by chimeric oligonucleotide correction, not by spontaneous mutation. Due to its high sensitivity, DOPA reaction could detect gene correction of one or two melanocytes per hair follicle. On the other hand, hair pigmentation may require correction of many melanocytes per hair follicle to produce and deposit enough melanin. Active tyrosinase was detected in skin sections 3 months after the last treatment with RDO. This prolonged expression of tyrosinase activity indicate that gene correction occurred in precursor melanocytes in the hair follicle, and that correction has lasted for more than one hair cycle.

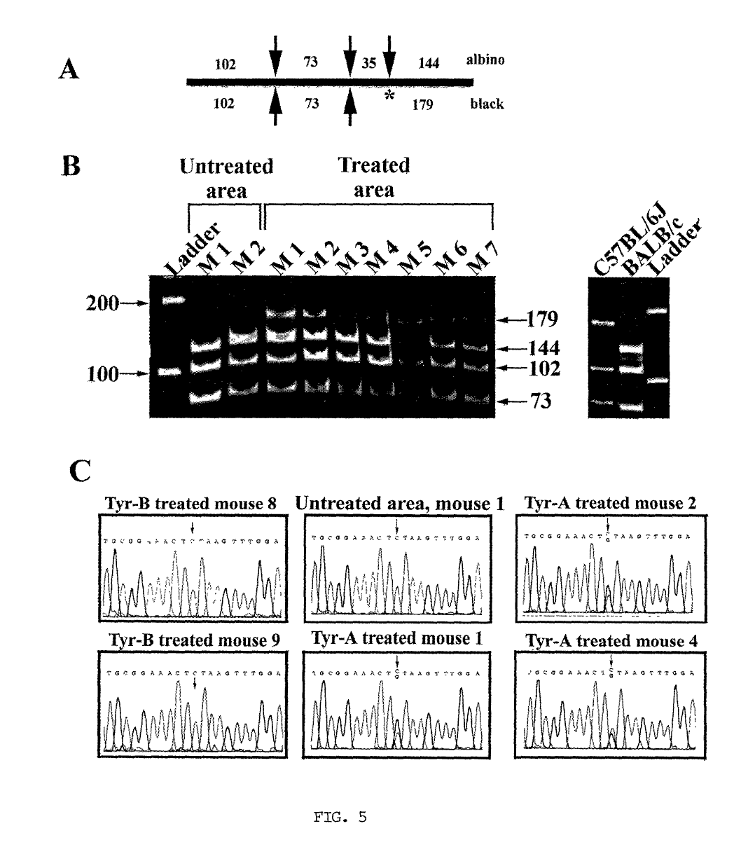

In order to verify the correction of the tyrosinase gene at the DNA level, genomic DNA was isolated from the same DOPA positive and negative skin biopsies from seven treated mice. Genomic DNA was subjected to PCR amplification using two primers, 5′-AAGAATGCTGCCCACCATG-3′ and 5′-GACATAGACTGAGCTGATAGTATGTT-3′, to generate a 354 bp fragment. The PCR product of genomic DNA from untreated albino skin, containing inactive tyrosinase (CTAAG) resulted in 144, 102, 73 and 35 bp fragments upon DdeI digestion (FIGS. 5A and B). In comparison, the PCR product of genomic DNA from the Tyr-A treated skin generated 179, 144, 102, 73 and 35 bp fragments upon DdeI digestion. The presence of 179 bp fragment upon DdeI digestion indicated a corrected tyrosinase gene (GTAAG). Surprisingly, a high level of gene correction was observed according to the intensity of the 179 bp fragment among biopsies taken from seven different mice (FIG. 5B). The DNA sequencing of the 354 bp PCR fragment from the Tyr-A treated skin also exhibited mixture of C and G nucleotides, indicating tyrosinase gene correction (FIG. 5C). No other sequence alterations within the 400 bp region were detected in any of the mice examined.

Surprisingly, a high level of gene correction approaching 40% was observed from skin biopsy of animal, in which RDO was injected intradermally or topically applied. Because skin sample contains both epidermis and dermis, this result indicates an efficient tyrosinase gene correction, not only in melanocytes but also in the other cells, such as keratinocytes and fibroblasts. Such a high frequency of gene correction observed in vivo contrasts to a low frequency measured in tissue cultured cells, primary keratinocytes and fibroblasts. Repetitive intradermal and topical applications may also facilitate a high level of gene conversion observed in this study. Many other methods of skin delivery, liposome and cream can be utilized for the delivery of RDO.

Frequency of gene correction demonstrated by the phenotype change, the number of dark pigmented hairs or the number of DOPA positive hair follicles (FIGS. 3 and 4), appears to be lower than the frequency of chromosomal DNA sequence correction observed from the same skin biopsy (FIG. 5). This could be explained in part that transcription of the tyrosinase gene is highly tissue-specific and hair-cycle dependent and takes place in hair follicle associated melanocytes. Thus, a phenotypic change would be caused exclusively by corrected melanocytes actively transcribing the tyrosinase gene. However, the skin biopsy contains both epidermis and dernis comprised of keratinocytes, fibroblasts, and melanocytes. In fact, melanocytes are only minor portion (<1%) of total population of cells present in the skin biopsy. Whereas genotypic change for the tyrosinase is measured in all cells in skin biopsy, including keratinocytes and fibroblaste. Tyrosinase gene correction in keratinocytes or fibroblasts would not result in pigmentation change but would result in genomic correction. The scarcity of melanocytes, and cycling expression of tyrosinase in the skin may in part explain such discrepancy. It is also possible that hair pigmentation may require correction of many melanocytes per hair follicle to produce and deposit enough melanin in hair shaft. In this case, gene correction of one or two melanocytes per hair follicle may not be detected as phenotypic change. High frequency of gene correction in vivo contrasts to a low frequency measured in tissue cultured cells, primary keratinocytes (Santana, et al., 1998, J Invest Dermatol, 111: 1172-1177).

In the present invention permanent gene correction by RDO did last during the life span of corrected cells and their progeny cells. Gene correction by RDO does not appear to require DNA replication, since gene conversion by an RDO was observed in quiescent Gₒ hepatocytes and CD34+-enriched cells (Xiang et al., 1997 J. Mol. Med., 75:829-835). The prolonged expression of tyrosinase activity lasting several hair cycles observed in this experiment indicates that gene correction occurred in precursor melanocytes which propagated their gene correction to daughter cells. The fact that gene correction has been observed in daughter cells shows that the correction (modification) occurred in a stable manner.

Collectively, these results demonstrate stable tyrosinase gene correction by RDO at the phenotypic and genomic levels in vivo. Efficient gene correction by RDO in skin suggests that this strategy is feasible for treating hereditary skin diseases resulting from a point mutation.

In another embodiment, reversal of hair loss by correction of the Desmoglein 3 gene in the balding mouse is described. Desmoglein 3 (Dsg3) is a desmosomal transmembrane glycoprotein that belongs to the cadherin superfamily of cell adhesion receptors and important in maintaining cell-to-cell adhesion (Amagai et al., 1991, Cell 76:869-877). Dsg-specific antibodies found in pemphigus patient's bind to the cell surface of keratinocytes and induce loss of cell adhesion leading to the formation of blisters (Amagai et al., 1992, J. Invest. Dermatol., 106:351-355). DSG3−/− mice developed oral lesions preventing normal food intake and suprabasilar acantholysis of skin when traumatized, similar to pemphigus patients (Koch et al., 1997). Furthermore, these mice developed hair loss, indicating its function in anchoring hair to the outer root sheath of the hair follicle (Koch et al., 1998, J. Cell Sci. 111:2529-2537).

The runting and hair loss phenotype of the DSG3−/− mice is identical to that of a previously reported mouse mutant, balding (bal) (Sunberg, 1994, In: Animal models and biomedical toods. J. P. Sundberg (ed), CRC Press Boca Raton, pp. 187-191). The mutation in bal/bal mice has been identified as one base insertion (2275insT) in exon 14 of DSG3 gene, which results in a frame-shift and a premature stop codon (Koch et al., 1997, J. Cell Biol., 137:1091-1102). Since it is a recessive mutation, correction of single allele will be sufficient for the reversal of phenotype. Correction of base deletion/insertion by RDO has not yet been shown in tissue culture or in vivo. Therefore, this system will provide us to test the efficacy of RDO in correction of single base insertion/deletion.

The bal/bal can be obtained from the Jackson Laboratory and RDO can be introduced to skin by any of the methods known in the art or disclosed herein. Repetitive treatments can be applied to the central dorsal area close to the forehead of mice, between d 1 and d 20 after birth, before the onset of the first telogen stage. Hair retention can be monitored continuously. Once telogen hair retention is observed, skin biopsy is taken. In order to investigate the permanence of gene correction, skin biopsy should be taken at various times. The expression of Dsg3 can be confirmed by Western blot analysis, using anti-FLAG M2 antibody and anti-Dsg3 antibody. Chromosomal gene correction can be determined by isolation of DNA from skin biopsy, Southern Blot analysis, and DNA sequencing. At the same time, histological characterization of skin, hair morphology, hair cycle analysis, and immunolocalization of Dsg3 can be carried out by using anti-FLAG M2 and anti-Dsg3 antibody.

The DSG3-A RDO contains a sequence homology of 30 base (24 RNA and 6 DNA residues) with a single base deletion in the center. Several other RDOs can be designed. Targeting a base pair insertion/deletion may require a longer length of DNA: longer stretch of RNA (24-32 residues) and DNA (6-10 residues) can be tested. A DNA mismatch repair may preferentially occur at a mismatch residing in the DNA:DNA duplex, rather than a mismatch in the RNA:DNA duplex. The longer and complete homology in RNA can be more efficient in the strand-pairing event driven by RNA. A strand-specific repair can be investigated by comparing the conversion frequencies of RDOs containing a single deletion, either in the DNA-containing strand (DSG3-B) or the RNA-containing strand (DSG3-C) of RDO.

```
                                            bal bal sequence
      5′ ATTCCACTGGAGGGACCTTGAAGGACTTATGCTGCGCCAGTCAACATGACTT 3′
                    TGCGCG-cccuggaacuucCTG-ATAcgacgcggucagT
```

| | |
|---|---|
| T | T DSG3-A |
| T | T |
| TCGCGC IGGGACCTTGAAGGAC-TATGCTGCGCCAGTCT | |
| 3′I5′ | |
| 3′ TAAGGTGACCTCCCTGGAACTTCCTGAATACGACGCGGTCAGTTGTACTGAA 5′ | |
| TGCGCG-cccuggaacuuccugaauacgacgcggucagT | |
| T | T DSG3-B |
| T | T |
| TCGCGC IGGGACCTTGAAGGAC-TATGCTGCGCCAGTCT | |
| 3′I5′ | |
| TGCGCG-cccuggaacuuccug-auacgacgcggucagT | |
| T | T DSG3-C |
| T | T |

```
                    TCGCGC IGGGACCTTGAAGGACTTATGCTGCGCCAGTCT
                              3′I5′
Each RNA residue, shown in lower case is modified by the inclusion of a 2′-O-methyl group on the ribose sugar. DNA residues are shown in upper case. The hyphen ("-") in the above sequence indicates contiguity of nucleotides without any gap. The vertical line ("I") indicates the beginning of the 5′ end and the ending of 3′ end.
```

In another aspect of the invention, the methods for the treatment of genetic disease epidermolysis bullosa (EB) is described. The group of inherited blistering skin diseases, collectively known as epidermolysis bullosa, are manifested due to mutations in the genes expressed in the cutaneous basement membrane zone primarily by basal keratinocytes.

EB can be divided into three major categories, based on the level of tissue separation within the cutaneous basement membrane zone, simplex, junctional and dystrophic. In the simplex forms, the tissue separation occurs within the basal keratinocytes as a result of mutations in keratin 5 and 14 genes. In the junctional forms of EB, the tissue separation occurs within the basement membrane itself, at the level of lamina lucida, and mutations in the genes encoding the anchoring filament protein laminin 5 (LAMB3, LAMB3 and LAMC2), bullous pemphigoid antigen 2 (BPAG2), and p4 chain of the integrin (ITGB4) have been delineated. In the dystrophic, scarring forms of EB, the diagnostic hallmark is abnormalities in the anchoring fibrils, attachment structures extending from the basement membrane to the underlying dermis. Anchoring fibrils are composed predominantly of type VII collagen.

Recently, a large number of mutations in the gene encoding type VII collagen (COL7A1) have been disclosed in different forms of dystrophic EB (Christiano et al., 1996, Advances in Dermatology 11, 199-213). Most of these mutations in recessive dystrophic EB are shown to be an incorporation of a premature stop codon by point mutation or frameshift in the coding region of the COL7A1 (Christiano et al., 1996, Advances in Dermatology 11, 199-213). In the dominantly inherited forms of EB, the recurrent mutation detected is the substitution of a glycine residue, which occurs within the collagenous domain of the Gly-X-Y repeating sequence (Christiano et al., 1996, Advances in Dermatology 11, 199-213). These amino acid substitutions destabilize the collagen triple helix and render the molecules susceptible to intracellular degradation. Some of these nonfunctional molecules can associate with type VII collagen synthesized from the normal allele, producing the blistering phenotype through a mechanism known as dominant negative interference. Because type VII collagen is a homotrimer consisting of three identical α1 (VII) polypeptides, only one of eight triple-helical molecules is normal, if there is equal expression of the mutant and wild-type alleles. As a result, some normal anchoring fibrils can be formed, consistent with ultrastructural demonstration of thin anchoring fibrils and relatively mild clinical phenotype.

Chimeric oligonucleotide can be used to create or to correct genetic mutations found in the junctional and the dystrophic EB. The followings are examples of chimeric oligonucleotides which are targeted to correct or to create a point mutation or a frameshift mutation of the type VII collagen and the BPAG2 genes found in EB patients.

(i) Correction of chromosomal mutations present in the dystrophic EB: human
type VII collagen
(a) Chimeric oligonucleotide designed to change the stop codon to Gln in
type VII collagen

| AGC CAA TCC TTG AGA GTA CAG TGG ACA GCG GCC AGT GGC | normal |
|---|---|
| TCG GTT AGG AAC TCT CAT GTC ACC TGT CGC CGG TCA CCG | |
| Ser Gln Ser Leu Arg Val Gln Met Thr Ala Ala Ser Gly | |
| AGC CAA TCC TTG AGA GTA TAG TGG ACA GCG GCC AGT GGC | Dystrophic EB |
| TCG GTT AGG AAC TCT CAT ATC ACC TGT CGC CGG TCA CCG | |
| stop | |
| T GCGCG agg aac ucu cAT GTC acc ugu cgc c T | |
| T T | correction |
| T T | (stop to Gln) |
| T CGCGC\|TCC TTG AGA GTA CAG TGG ACA GCG G T | |
| 3′5′ | |
| T GCGCG agg aac ucu cAT TTC acc ugu cgc c T | |
| T T | mutation |
| T T | (Gln to stop) |
| T CGCGC\|TCC TTG AGA GTA AAG TGG ACA GCG G T | |
| 3′5′ | |

(b) Chimeric oligonucleotide designed to correct or create a dominant
negative mutation (Gly to Ser) of the human type VII collagen at amino acid
residue 2040 of the type VII collagen.

| | |
|---|---|
| GGG GAG CCT GGA AAG CCT GGT ATT CCC GGG CTC CCA GGC | normal |
| CCC CTC GGA CCT TTC GGA CCA TAA GGG CCC GAG GGT CCG | |
| Gly Glu Pro Gly Lys Pro Gly Ile Pro Gly Leu Pro Gly Gly | |
| GGG GAG CCT GGA AAG CCT AGT ATT CCC GGG CTC CCA GGC | Dominant |
| negative EB | |
| CCC CTC GGA CCT TTC GGA TCA TAA GGG CCC GAG GGT CCG | |
| Ser | |
| T GCGCG gga ccu uuc gGA TCA uaa ggg ccc g T | |
| T T | Correction |
| T T | (Ser to Gly) |
| T CGCGC\|CCT GGA AAG CCT AGT ATT CCC GGG C T | |
| 3′5′ | |
| T GCGCG gga ccu uuc gGA TCA uaa ggg ccc g T | |
| T T | Mutation |
| T T | (Gly to Ser) |
| T CGCGC\|CCT GGA AAG CCT AGT ATT CCC GGG C T | |
| 3′5′ | |

(ii) Chromosomal mutation in the junctional EB: human BPAG2 gene
(a) Chimeric oligonucleotide designed to change the stop codon to Arg

| | |
|---|---|
| CCT GGT CCC CCA GGG CCT CGA GGG CCC CCG GGT GTC TCA | normal |
| GGA CCA GGG GGT CCC GGA GCT CCC GGG GGC CCA CAG AGT | |
| Arg | |
| CCT GGT CCC CCA GGG CCT TGA GGG CCC CCG GGT GTC TCA | Junctional EB |
| GGA CCA GGG GGT CCC GGA ACT CCC GGG GGC CCA CAG AGT | |
| stop | |
| T GCGCG ggg ggu ccc gGA GCT ccc ggg ggc c T | |
| T T | correction |
| T T | (Stop to Arg) |
| T CGCGC\|CCC CCA GGG CCT CGA GGG CCC CCG G T | |
| 3′5′ | |
| T GCGCG ggg ggu ccc gGA ACT ccc ggg ggc c T | |
| T T | mutation |
| T T | (Arg to stop) |
| T CGCGC\|CCC CCA GGG CCT TGA GGG CCC CCG G T | |
| 3′5′ | |

(b) Chimeric oligonucleotides designed to create a dominant negative mutation
CGC (Arg) to CCC (Pro) in the human KRT14 gene and to correct the same mutation in
the gene

| | |
|---|---|
| ATG CAG AAC CTC AAT GAC CGC CTG GCC TCC TAC CTG GAC | wt |
| TAC GTC TTG GAG TTA CTG GCG GAC CGG AGG ATG GAC CTG | |
| Arg | |
| T CGTCT ug gag uua cug gcg gac cgg agg au T | make mutation |
| T T | HKRT14-A |
| T T | (Arg to Pro) |
| T GCAGA\|AC CTC AAT GAC CCC CTG GCC TCC TA T | |
| 3′5′ | |
| OR | |
| U CGTCT ug gag uua cug gcg gac cgg agg au U | to make mutation |
| U U | HKRT14-B (modified hair pin loops) |
| U U | (Arg to Pro) |
| U GCAGA\|AC CTC AAT GAC CCC CTG GCC TCC TA U | |
| 3′5′ | |
| T CGTCT ug gag uua cug ggg gac cgg agg au T | to correct mutation |
| T T | HKRT14-C |
| T T | (Pro to Arg) |
| T GCAGA\|AC CTC AAT GAC CGC CTG GCC TCC TA T | |
| 3′5′ | |
| OR | |
| U CGTCT ug gag uua cug ggg gac cgg agg au U | |
| U U | HKRT14-D (modified hair pin loops) |
| U U | (Pro to Arg) |
| U GCAGA\|AC CTC AAT GAC CGC CTG GCC TCC TA U | |
| 3′5′ | |

Each RNA residue, shown in lower case is modified by the inclusion of a 2′-O-methyl group on the ribose sugar.
DNA residues are shown in upper case. The vertical line (“|”) indicates the beginning of the 5′ end and the ending of 3′ end.

The methods of the present invention can also be used to treat acquired skin diseases. As a specific embodiment of the invention, strategies to treat psoriasis is explained here. Psoriasis represents an inflammatory skin disorder characterized by loss of normal cellular homeostasis, resulting in epidermal hyperproliferation, defective differentiation and inflammation.

In psoriatic epidermis, T helper and T suppresser lymphocyte infiltrates psoriatic lesion with marked increase in the number of macrophages. The IL-I released from macrophages or epidermal cells upon their activation induces T cells to IL-2 production and expression of surface IL-2 receptor. These cytokines involve both proximal steps in the activation of lymphocytes and the epidermal hyperproliferation.

The chimeric RDO technology is used for the treatment of psoriasis by inactivating cytokines or cytokine receptors implicated in psoriasis, such as IL-1α/β, IL-2 or IL-2 receptor, and TNFα gene Other targets involve adhesion molecules, such as ICAM and VCAM. Several approaches can be utilized for inactivation of harmful proteins : (i) conversion of a codon for amino acid (XAA, XAG and XGA, where X can be A, G, or C) to a stop codon (TAA, TAG and TGA) by a single base change, (ii) conversion of a splice junction sequence (AG or GT) to a sequence which will inactivate the splicing event by a single base change, (iii) targeted mutagenesis of an important amino acid residue to render a protein non-functional (iv) alteration of an amino acid to make a trans-dominant mutant protein which interferes with an assembly of multiple subunits of proteins (for example, collagen type I), or which binds to the receptor with a high affinity but lost a capability for a signal transduction (ligand-receptor interaction), or which acts competitively to reduce the ability of viral proteins (HIV proteins, tat and rev) to interact with crucial effector molecules. For example, the following chimeric oligonucleotides can be synthesized and used for the treatment of psoriatic skin disorders. These chimeric oligonucleotides can be used either singly or in combination.

(a) Chimeric oligonucleotide designed to introduce the stop codon in the IL-1β gene

EXON 1 (930 GAA to TAA) Glu to Stop
TCT GAA GCA GCC ATG GCA GAA GTA CCT GAG CTC
AGA CTT CGT CGG TAC CGT CTT CAT GGA CTC GAG

| T CGCGC cgu cgg uac cGT ATT cau gga cuc g T | |
|---|---|
| T T | IL-1β(A) |
| T T | |
| T GCGCG\|GCA GCC ATG GCA TAA GTA CCT GAG C T | |
| 3′5′ | |

(b) Chimeric oligonucleotide designed for a splice junction (GT to
TT) in the IL-1β gene
EXON 1 / INTRON 1
GAA ATG ATG GCT TAT TAC AG/GTC AGT GGA GAC GCT GAG ACC
CTT TAC TAC CGA ATA AGG TC/CAG TCA CCT CTG CGA CTC TGG

| | |
|---|---|
| T CGCGC c cga aua aug TC AAG uca ccu cug cT | |
| T T | IL-1β(B) |
| T T | |
| T GCGCG\|G GCT TAT TAC AG TTC AGT GGA GAC GT | |
| 3′5′ | |

(c) (Chimeric oligonucleotide designed to generate a mutant IL-1β
that binds to the receptor but prevents signal transduction (Arg 11
to Gly 11: CGA to GGA)
4 5 6 7 8 9 10 11 12 13 14 15 16 17 aa of the
mature IL-1β
Arg Ser Leu Asn Cys Thr Leu Arg Asp Ser Gln Gln Lys Ser
CGA TCA CTG AAC TGC ACG CTC CGG GAC TCA CAG CAA AAA AGC
GCT AGT CAC TTG ACG TGC GAG GCC CTG AGT GTC GTT TTT TCG

| | |
|---|---|
| T CGCGC uug acg ugc gAG CCC cug agu guc g T | |
| T T | IL-1β(C) |
| T T | |
| T GCGCG\|AAC TGC ACG CTC GGG GAC TCA CAG C T | |
| 3′5′ | |

(d) Chimeric oligonucleotide designed to introduce the stop codon in
the TNFα gene (AAG to TAG)
Exon 1
GAG GAG GCG CTC CCC AAG AAG ACA GGG GGG CCC CAG GGC
CTC CTC CGC GAG GGG TTC TTC TGT CCC CCC GGG GTC CCG

| | |
|---|---|
| T GCGCG cgc gag ggg uTC ATC ugu ccc ccc g T | |
| T T | TNFα |
| T T | |
| T CGCGC\|GCG CTC CCC AAG TAG ACA GGG GGG C T | |
| 3′5′ | |

(e) Chimeric oligonucleotide designed to introduce the stop codon in
the IL-1α gene (TGG to TAG)
Exon 3
TGT GAC CCA CAA CTA TCA TGG TCA TTA AAG TAC ATT GGC CAG
ACA CTG GGT GTT GAT AGT ACC AGT AAT TTC ATG TAA CCG GTC

| | |
|---|---|
| T GCGCG gu guu gau agT ATC Agu aau uuc au T | |
| T T | IL-1α |
| T T | |
| T CGCGC\|CA CAA CTA TCA TAG TCA TTA AAG TA T | |
| 3′5′ | |

Each RNA residuem shown in lower case, is modified by the inclusion of a 2′-O-methyl group on the ribose suger. DNA residues are shown in upper case. The vertical line (“|”) indicates the beginning of the 5′ end and the ending of 3′ end.

The pharmacokinetics of oligonucleotides in vivo can be measured to improve upon its design and delivery. Stability and uptake of antisense oligonucleotides have been studied extensively in tissue culture cells. In comparison, relatively little information is available concerning the in vivo stability and tissue localization of antisense oligonucleotides. A simple nonradioactive method can be used to quantitate oligonucleotides in biological specimens. This method is based on extraction of the oligonucleotide from the biological fluids or tissues, followed by immobilization on a nylon membrane. The membrane bound oligonucleotide is then hybridized with the labelled complementary oligonucleotide. A standard curve is generated by loading a known amount of oligonucleotide to estimate the amount of oligonucleotide present in biological specimens. Skin is extracted with phenol/chloroform, denatured, blotted on the nylon membrane and hybridized to a radiolabelled complementary single-stranded oligonucleotide. Tissues from lung, kidney, liver, muscle, brain, heart, spleen and blood are also used to estimate the amounts of the chimeric oligonucleotide in these tissues. Determination of the tissue distribution should address not only the efficacy, but also the biosafety issue of the chimeric oligonucleotide pertinent towards development of this gene therapy aspect for human application in the future.

The method described above estimates the total quantities of chimeric oligonucleotide, both intact and degraded forms. In order to assess the stability of chimeric oligonucleotides in vivo, skin from the injected area and uninjected area (control) are phenol/chloroform extracted, ethanol precipitated, 5′-labelled with ³²P γ-ATP and loaded on polyacrylamide gel containing 7 M urea. Degradation of chimeric oligonucleotides are determined according to the size of fragments generated. It has been found that this method correlates well with the in vivo study where animal was injected with an oligonucleotide radiolabelled internally.

Morphology of the skin of the mouse is monitored and compared between the areas where the chimeric oligonucleotide causing a negative dominant mutation and the control oligonucleotide are applied: blisters, crusted erosions, scar formation, exuberant granulation of tissue, pigmentation changes. Each graft is removed from mouse, snap frozen, paraffin-embedded and stained with hematoxylin and eosin for microscopic examination. If there is a change in the morphology, a further characterization can be carried out on the ultrastructural level by transmission electron microscopy. Different forms of EB (simplex, junctional and dystrophic) is determined by direct visual assessment of basilar keratinocyte tonofilaments and by examining the number and appearance of hemidesmosomes and anchoring fibrils. Reduced number of the anchoring fibrils, attachment structures extending from the basement membrane to the underlying dermis, is a diagnostic hallmark of dominant dystrophic EB.

An indirect immunofluorescence that uses the known ultrastructural localization of several skin basement membrane proteins, including BPAG1, BPAG2, laminin 5 and type VII collagen, can be carried out to predict the location of skin cleavage within a given skin specimen. A dominant negative mutation in type VII collagen is indicated by a reduction in anchoring fibril and in the amounts of the collagen type VII. Graft is removed from the mouse and will be snap-frozen in isobutanol and liquid nitrogen. Section is embedded using Tissue-Tek O.C.T. compound (Miles, Elkhart, Ind.) and cut into 5 micron sections on a cryostat. Immunofluorescence can be performed using, for example, VectaStain ABC kit (AK-5002 alkaline phosphatase) (Vector Laboratories, Burlingame, Calif.) with collagen type VII polyclonal antibodies (Sigma, St. Louis, Mo.) following manufacturer's protocol.

In another aspect of the present invention a specific mutation in murine skin can be generated by in situ application of RDO. Point mutations found in the dominant forms of simplex and dystrophic human EB can be created in the corresponding mouse genes. RDO can be introduced to mouse by developing several modalities of in vivo delivery described herein. For phenotypic change, a localized skin blistering can be monitored. Skin biopsy can be taken for histological analysis, protein, and DNA sequence determination.

After the application of the appropriate RDO to create a mutation in an EB gene in a localized area of a murine skin, the murine skin morphology can be monitored for blisters, crusted erosions, millia, scar formation, exuberant granulation of tissue or pigmentation changes, scarring alopecia and dystrophy or absence of nails. Skin sections can be removed from mice, snap frozen or paraffin-embedded and stained with hematoxylin and eosin for microscopic examination. If there is a change in the morphology, a further characterization can be carried out on the ultrastructural level by transmission electron microscopy. Skin cleavage at ultrastructural level, epidermolytic, lamina lucidolytic or dermolytic can be examined. In addition, basilar keratinocyte tonofilaments (SEB), number and appearance of hemidesmosomes (JEB), presence of anchoring filaments and subbasal dense plates (severer forms of JEB), number and appearance of anchoring fibrils (DEB) and the presence and extent of upper dermal collagenolysis can be examined (DEB).

An indirect immunofluorescence of proteins can be carried out to predict the location of skin cleavage within a given skin specimen. Skin sections can be removed from the mouse and be snap-frozen in isobutanol and liquid nitrogen. Skin section is embedded using, for example, Tissue-Tek O.C.T. compound (Miles, Elkhart, IN) and cut into 5μ sections on a cryostat. Immunofluorescence can be performed using, for example, VectaStain ABC kit (AK-5002 alkaline phosphatase) (Vector Laboratories, Burlingame, CA) with collagen type VII and keratin 14 antibody. Western blot analysis can be carried out to determine the size of the protein expressed in mouse skin. In addition, genomic DNA from skin biopsy can be isolated as described (Alexeev et al., 1999) and DNA sequence can be analyzed by PCR-based RFLP, Southern blot, and DNA sequencing.

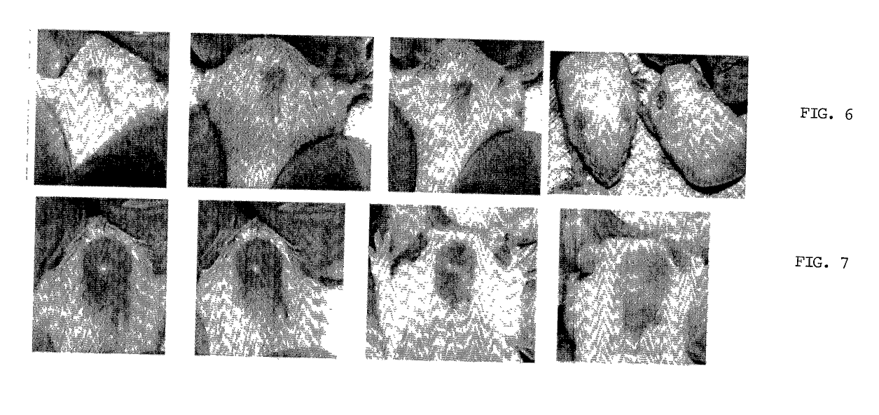

The efficacy of gene conversion by RDO by creating a dominant mutation in KRT17 and COL7A1 gene in a localized area of murine skin was tested. In order to generate a dominant mutation, BALB/c mice was treated with RDO at day 1, 3, 5 after birth repetitively, prior to the hair growth. In these mice, a genotypic change caused by RDO resulted in a phenotypic change, a localized skin blisteriaround day 7after birth. Shown in FIGS. 6 and 7 are mice carrying dominant mutations in the COL7A1 (FIG. 6) and KRT17 (FIG. 7) genes. In both cases, mice developed blisters whereas in the control (treated with the chimeric oligonucleotide fully complementary to the target sequence and/or cytofectin only) no such blisters were observed.

The design synthesis and purification of RDO for this experiment were as follows:

Two RDOs were designed to make a dominant mutation in mouse KRT17 and COL7A1 gene, respectively. The sequence was determined by comparing the sequence of corresponding human genes and by making an analogous mutation found in the most severe and dominant forms of SEB and DEB patients. Substitution of Arg to Pro in the exon 1 of KRT17 gene caused the most severe form of SEB, while substitution of Gly to Ser in the triple helical repeat region of COL7A1 caused a dominant form of JEB. The structure of RDO was modified from the original design according to the inventor's recent findings. It was found that the length and the complete homology of RNA to the target sequence and chemical modification of the hairpin loop were important to improve the gene correction activity of RDO. Furthermore, it was found that placement of a single mismatch in the DNA containing strand of RDO was important to induce a preferential DNA mismatch repair. Modification of RDO resulted in approximately 10-fold higher activity than the original design. Synthesis and purification has been carried out as described (Yoon et al, 1996, Proc. Natl. Sci. Acad. 93:2071-2076). RDO was applied to skin by intradermal injection.

(a) RDO designed to create a dominant negative mutation CGC (Arg) to CCC (Pro) in the mouse KRT17gene

| ATG CAG AAC CTC AAT GAC CGC CTG GCC TCC TAC CTG GAC | wt |
|---|---|
| TAC GTC TTG GAG TTA CTG GCG GAC CGG AGG ATG GAC CTG | |
| T CGTCT ug gag uua cug gcg gac cgg agg au T | |
| T T | MKRT17-A |
| T T | (Arg to Pro) |
| T GCAGA\|AC CTC AAT GAC CCC CTG GCC TCC TA T | |
| 3′5′ | |
| OR | |
| T CGTCT ug gag uua cug gcg gac cgg agg au U | |
| T U | MKRT17-B |
| T U | (Arg to Pro) |
| T GCAGA\|AC CTC AAT GAC CCC CTG GCC TCC TA U | |
| 3′5′ | |
| (b) Chimeric oligonucleotides (RDOs) designed to create a dominant negative | |
| mutation CGC (Gly) to AGC (Ser) in the mouse type VII collagen (COL7A1) at amino | |
| acid residue 2040 and to correct the same mutation in the gene | |
| GCGGGG GAA CCC GGA AAG CCT GGC ATT CCT GGA CTC CCA GGC CGG | wt |
| CGCCCC CTT GGG CCT TTC GGA CCG TAA GGA CCT GAG GGT CCG GCC | |
| Gly | |
| T CCCTT ggg ccu uuc gga ccg uaa gga ccu g T | make mutation |
| T T | MCOL7-A |
| T T | (Gly to Ser) |
| T GGGAA\|CCC GGA AAG CCT AGC ATT CCT GGA C T | |
| 3′5′ | |
| OR | |
| U CCCTT ggg ccu uuc gga ccg uaa gga ccu g U | make mutation |
| U U | MCOL7-B (modified hair pin loops) |
| U U | (Gly to Ser) |
| U GGGAA\|CCC GGA AAG CCT AGC ATT CCT GGA C U | |
| 3′5′ | |
| T CCCTT ggg ccu uuc gga ucg uaa gga ccu g T | correct mutation |
| T T | MCOL7-C |
| T T | (Ser to Gly) |
| T GGGAA\|CCC GGA AAG CCT GGC ATT CCT GGA C T | |
| 3′5′ | |
| OR | |
| U CCCTT ggg ccu uuc gga ucg uaa gga ccu g U | correct mutation |
| U U | MCOL7-D (modified hair pin loops) |
| U U | (Ser to Gly) |
| U GGGAA\|CCC GGA AAG CCT GGC ATT CCT GGA C U | |
| 3′5′ | |

Shown above are the sequences of the RDO and the targeted sequences where the target site in the target sequence is underlined in the sequence. DNA residues are capitalized and the 2′-O-methyl RNA residues are in lower case. The vertical line (“|”) indicates the beginning of the 5′ end and the ending of 3′ end.

Oligonucleotides composed of a contiguous stretch of RNA and DNA residues have been developed to facilitate correction of mutations in mammalian cells (see Yoon et al., 1999, Biogenic Amines, 15:137-167; U.S. Pat. No. 5,795,972). The first design of RNA-DNA oligonucleotide (RDO) or chimeric oligonucleotide contained 20 residues of RNA (Yoon et al., 1996; PNAS USA, 93:2071-2076).

For mammalian gene conversion experiments, it was necessary to make structural and chemical modifications of RDO. The RNA:DNA duplex was linked by a double-hairpin structure containing four T residues in each loop and a five base-pair GC clamp, to protect the 5′- and 3′-end of the oligonucleotide from exonucleolytic cleavage. This modification also prevented tandem ligation, which has been known to inactivate transfected DNA in mammalian cells. It was necessary to incorporate some modification to render the oligonucleotide resistant to the Rnase H and other RNases (Monia et al., 1992). The first of such attempt was a 2′-O-methylation of the ribose sugar. The 2′-O-methyl RNA exhibited a similar affinity to the DNA target as unmodified RNA or DNA.

The basic design of a chimeric oligonucleotide comprises of RNA and DNA residues (10 RNA, 5 DNA and 10 RNA), complementary to the central or other region of the coding strand of the targeted gene and configured into a double hairpin with four T residues on both ends. The DNA residue which participates in the mismatch repair is purposely placed in the center region of the RNA-bordered DNA-section. Overall, the molecule contains 3′ and 5′ unligated ends and each RNA residue is modified by the addition of a 2′-O-methyl group to the ribose sugar. Several variables of the basic design can be used which can differ in (i) length of RNA, (ii) required length of DNA residues bordering RNA for correction of a mismatch and a frameshift mutation, (iii) strand specificity for RNA residues, (iv) complementary sequence to the coding strand containing all RNA residues with no DNA interruption, and (v) vehicle with all RNA or chemically modified RNA residues, (vi) a double hairpin with four “U” (uridine) residues instead of “T” residue. For example, the length of RNA in a chimeric oligonucleotide can be preferably from 45-50 ribonucleotides, more preferably from 40-45 ribonucleotides, still more preferably from 30-40 ribonucleotides still most preferably from 25-30 or 20-30 ribonucleotides. In some embodiments the length can be 25-50 ribonucleotides.

Instead of DNA interruption, it is preferable to make one strand of chimeric oligonucleotide to contain all RNA sequence entirely complementary to one strand of targeted DNA. The other strand of chimeric oligonucleotide should contain all DNA sequence complementary to the other strand of targeted DNA,with an exception of one mismatch at the nucleotide position corresponding to the nucleotide position in the target gene sequence where genetic alteration will be made or with an exception of 1, 2 or 4 nucleotide deletions or insertions instead of one mismatch in the other strand. This 1, 2 or 4 nucleotide deletions or insertions in other strand is to create frameshift mutations in the target gene.

Chemical modification of the chimeric molecule (i.e., a chimeric RDO) backbone, sugar and base modifications, can be made to meet the following requirements: (i) base or backbone modification should not appreciably alter thermodynamic or kinetic parameters for association of two strands, (ii) modification should improve the delivery of oligonucleotide to the cellular environment and increase the resistance to nuclease degradation, and (iii) modifications should not alter cellular functions responsible for biological activity. Modifications of the heterocyclic bases offer an opportunity to enhance the affinity without compromising RNase H cleavage of the target RNA in an antisense mode. Hydrophobic modifications at the 5-position of pyrimidines, such as 2′-deoxyuridine, 5-fluoro-2′-deoxyuridine, 5-bromo-2′-deoxyuridine and 5-methyl-2′-deoxycytidine, can enhance thermodynamic stability toward RNA or DNA target (67). Various backbone modifications, such as phosphorothioates, phosphoramidites and methylphosphonates, and those with nonphosphate intemucleotide bonds, such as carbonates, carbamates, siloxanes, sulfonamides and polyamide nucleic acid (PNA), can have increased resistance to nucleases. Above of all, modification should not alter cellular functions responsible for biological activity, and in this case recombination and repair.

A chimeric molecule which contains sugar modification, 2′-O-methyl in the RNA, was the first attempt of chemical modification. The nature of the 2′ substitution is the primary chemical difference between DNA and RNA and is likely to play an important role in relative duplex and triplex stability. This modification also makes the chimeric oligonucleotide resistant to RNase H and other RNases. In other words, this and other such modifications afford protection from nucleases. Uniform 2′-O-methyl modification of the DNA strand increases the stability of DNA:RNA hybrids. Further, homologous DNA pairing activity occurs with an RNA/DNA chimeric molecule in which the 2′-O-methyl modification has been made.

The sugar, base and backbone modifications described above can be incorporated into the basic design of the chimeric oligonucleotides to make variants of the basic design. For backbone modification, available monomeric unit of β-cyanoethyl phosphorarnidites, methyl phosphoramidites, and H-phosphonates (Glen Research, VA) can be incorporated during synthesis of the chimeric oligonucleotide. For sugar and base modifications, monomeric base unit of 2′-OMe-RNA-β-cyanoethyl phosphoramidites, 5′-fluro-2′-OMe-cytosine monomers and 2′-OMe-2-aminopurine-p-cyanoethyl phosphoramidite (Glen Research, VA) are incorporated for modification of pyrimidine and ribose ring. The chimeric oligonucleotides can be synthesized on a 0.2 μmole scale using the 1000 A wide pore CPG on the ABI 392 DNA/RNA synthesizer. The exocyclic amine groups of DNA phosphoramidites (Applied Biosystems, Foster City, Calif.) are protected with benzoyl for adenine and cytidine, and with isobutyryl for guanine. The 2′-O-methyl RNA phosphoramidites (Glen Research, Sterling, Va.) are protected with phenoxyacetyl group for adenine, dimethylformamidine for guanine and isobutyryl for cytidine. After the synthesis is complete, the base-protecting groups are removed by heating in ethanol: concentrated ammonium hydroxide (1:3), for 20 h at 55° C. The crude oligonucleotides can be purified by polyacrylamide gel electrophoresis. The entire oligonucleotide sample can be mixed with 7 M urea and 10% glycerol, heated to 70° C. and loaded on a 10% polyacrylamide gel containing 7 M urea. After gel electrophoresis, DNA bands can be visualized by UV shadowing, dissected from the gel, crushed and eluted overnight in TE buffer (10 mM Tris-HCl and 1 mM EDTA, pH 7.5) with shaking. The eluent containing gel pieces can be spun through 0.45 μm spin filter (Millipore, Bedford, Mass.) and precipitated with ethanol. Samples will be further desalted by G-25 spin column (Boehringer Mannheim, Indianapolis, Ind.).

Accessibility of the epidermis makes it feasible to deliver RDO directly to the skin by intradermal and topical application of the RDO/liposome complex. Furthermore, RDO is much more stable than the plasmid. Thus, a slow and sustained release of RDO in vivo can be considered in combination with the polymer matrix. The fluorescein-conjugated RDO (FITC-RDO) and fluorescent microscopy can be used to determine efficient delivery method. A number of in vivo delivery methods that are specifically described herein and that are known to those skilled in the art can be used to introduce the RDO into the skin. For example, emerging technologies of gene delivery systems, such as liposomes, receptor mediated endocytosis and particle bombardment (gene gun) strategy can be utilized for the in vivo as well ex vivo delivery of RDO.

In addition to intradermal injection and topical application methods, a sustained in vivo delivery by polymer matrices can be used. Copolymers of D, L-lactide and glycolide can be mixed at 85:15 (w/w) ratio and grounded to a particle size ranging from 100 to 250 μm. For example, various amounts of Tyr-A RDO (10-200 μg) are mixed with 40 mg PLG, frozen with liquid nitrogen, and lyophilized. The resulting disc is allowed to equilibrate within a high-pressure CO₂. Rapid reduction of pressure causes the polymer particles to expand and fuse into an interconnected structure. The PLG sponge imbedded with the FITC-RDO can be implanted to mice subcutaneously. Release and distribution of RDO in skin can be determined at various times by histological characterization of skin biopsy by confocal microscopy.

Another method that can be used is a gene gun delivery method. The gene gun accelerates DNA-coated gold particles into target cells or tissues. Due to their small size the particles actually penetrate through the cell membrane, carrying the bound DNA into the cell where DNA dissociates from the gold particle and can be expressed. Since this method is cell receptor-independent, it can deliver genes into different tissues. The gene gun delivery of plasmid resulted in a localized and transient transgene expression, in epidermal and dermal layer of mouse skin (Lu et al, 1997).

In vivo electroporation is another method that can be used to deliver RDO into skin cells combining electroporation in vivo together with injection of plasmid DNA enhanced expression 100 fold higher than that of intramuscular DNA injection alone (Aihara and Miyazaki, 1998). By using various types of electrode, efficiency of gene transfer was further increased, resulting in 100-10,000 fold increase in expression (Mir et al., 1999). This technique has also been successful in other tissues such as liver, testis, and skin. A protocol has been developed for intradermal and topical application of RDO in mouse skin (Alexeev et al., 1999). At defined time after DNA injection (25 s to 1 min), two stainless steel plate electrodes will apply transcutaneous electric pulse, placed 4-5 mm apart in the dorsal mouse skin. Experiments can be performed with long electric pulses of lower voltage-to-distance ratio to increase DNA transfer, 200 V/cm and 50 ms per pulse generated by T820 electroporator (BTX, San Diego, Calif.). The in vivo uptake of RDO can be monitored using FITC-RDO and fluorescence microscopy.

The methods of in vivo delivery and are further explained below which can be used for all skin disorders described herein. The dose of each type of RDO can be from 10-200 μg. However, dose and duration of treatment is determined individually depending on the degree and rate of improvement. Such determinations can be performed routinely by those of skill in the art. The method of the present invention can be used to treat skin disorders in humans. A selected delivery system can be used to treat locally both genetic skin disorders and acquired skin diseases in humans. Further, the method can be used to correct or create skin disorders not only in mice but also other animals such as rabbits, monkeys, pigs, cows, goats and such other animals.

### EXAMPLES

The following examples further illustrate the present invention, but of course should not be constructed as in any way limiting its scope. The examples below are carried out using standard techniques, that are well known and routine to those of skill in the art, except where otherwise described in detail. The examples are illustrative, but do not limit the invention. All animal methods of treatment or prevention described herein are preferably applied to mammals, most preferably humans.

### Example 1

In vivo Skin Delivery of RDO.

Shown in FIG. 1 are Sequences of the Tyr-A RDO and the targeted sequences in the tyrosinase gene. The target site is underlined in the sequence. DNA residues are capitalized and the 2′-O-methyl RNA residues are in lower case. The oligonucleotide Tyr-A (A) contains 25 bp of sequence identical to the wild type tyrosinase whereas the control oligonucleotide Tyr-B (B) is identical to the albino tyrosinase. The Tyr-A RDO was designed to introduce a single base pair substitution TCT→TGT in tyrosinase gene in order to convert an unpigmented albino melanocytes to black-pigmented cells by introducing a single amino acid change, Ser→Cys at amino acid 85 of the mature tyrosinase (Alexeev, et al., 1998. Nat Biotech, 16: 1443-1346).

For in vivo skin delivery of RDO, 10 μg FITC-RDO was mixed with 10 μg Cytofectin™ (Glen Research, Sterling, Va.) in 50 μl of the OptiMEM™ (Gibco, Bethesda, Md.) for 30 min prior to application. The complexes were topically applied or intradermally injected by 30G needle to skin of 30 day old mice. Skin biopsies, 5×5 mm, were taken 6-8 h after injection and transferred to embedding mold filled with O.C.T. compound (Fisher Scientific, Pittsburgh, Pa.). Molds were rapidly submerged into 2-propanol cooled with liquid nitrogen and stored at −80° C. 7 μm sections were cut on a microtome (HM 500, Carl Zeiss, Inc., Thomwood, N.Y.) at −20° C. Slides were analyzed by fluorescence microscopy as described previously (Alexeev, et al., 1998. Nat Biotech, 16: 1443-1346).

The in vivo application of the Tyr-A and Tyr-B RDO to mouse skin was carried out by two different methods, intradermal injection and topical application.

Topical application: The RDO/Cytofectin complex (75 μg Cytofectinm: 15 μg RDO) was prepared in 50 μl of OptiMEM and applied to the back skin of mice 2 days after depilation with Neet™ each day for 3 days.

Intradermal administration: Two days prior to application, mice were depilated to induce anagen. Mice were injected with 50 μl of Tyr-A RDO/Cytofectin complex (10 μg Cytofectin: 10 μg RDO) in OptiMEM repetitively. Animals were sacrificed at 2-5 months after birth and skin biopsies (5 mm²) were taken from both treated and untreated areas. One half of the tissue was used for HE and DOPA staining and the other half was used for isolation of the genomic DNA.

Illustrated in FIG. 2 is the Deliveiry of the RDO in the mouse skin. (A) Confocal fluorescence micrograph of the frozen skin section of BALB/c mouse in which a fluorescein-conjugated RDO was applied topically and (B) injected intradermally to skin. Abbreviations used are ep, for epidermis; hf, for hair follicle; dm, for dermis. Efficiency of delivery was monitored by a fluorescein-conjugated RDO (RDO-FITC) using fluorescence microscopy as described above. The RDO-FITC was incubated either with Cytofectin™ or SuperFect™. The complex was either topically applied or intradermally injected into mouse skin and biopsy was taken 6-8 h after application. Both delivery methods were used in further experiment. Shown in FIG. 3 is the Localization of dark pigmented hairs in albino BALB/c mouse. (A) and (B) indicate hairs of two albino mice after topical application of the Tyr-A RDO. (C) skin of albino mice after topical application of the control Tyr-B RDO. (D) and (E) represent mouse coat of two mice after intradermal injection of the Tyr-A. (F) hairs of albino mice after intradermal injection of the Tyr-B RDO. (G), (H) and (I) are magnified views of a single hair shown in panels D, E, and F, respectively.

### Example 2

DOPA Staining and Histology

Skin biopsies were imbedded in OCT solution (Fisher Scientific, Pittsburgh, Pa.) and quickly frozen at −80° C. The 7 μm sections of skin treated with Tyr-A or Tyr-B were cut on a microtome (HM 500, Carl Zeiss, Inc., Thornwood, N.Y.) at −20° C. and slides were incubated in 0.1% L-DOPA (Sigma, Saint Louis, Mo.) solution in phosphate buffer, pH 7.3 at 37° C. for 2 h, changing DOPA solution every 30 min to avoid auto-oxidation. An average of 100-150 frozen sections were cut for each speciment from two animals treated with Tyr-A and two animal treated with Tyr-B. The same slides were stained with hematoxylin and eosin (Fisher Scientific, Pittsburgh, Pa.). Melanin was also detected using the Fontana-Masson stain (Jones et al., 1993, Cell 73:713-724) on formalin-fixed paraffin embedded tissue sections. Sections were counter stained with nuclear fast red.

Detection of tyrosinase activity and melanin in skin sections after intradermal injection of the Tyr-A and Tyr-B RDO is illustrated in FIG. 4. (A) and (B) show absence of tyrosinase activity by DOPA stain in frozen sections from two mice treated with control Tyr-B RDO. (C) and (D) present DOPA and HE staining of the section from untreated area of the mouse 1 indicating the absence of tyrosinase activity in the anagen stage of hair follicles. (E) and (F) show active tyrosinase by DOPA oxidation reaction in Tyr-A RDO treated area of the mouse 1 and HE staining of the same section. (G) and (H) DOPA staining of Tyr-A RDO treated area of the mouse 2. (I) and (J) detection of melanin by Fontana-Masson stain of the Tyr-A treated and Tyr-B treated areas from the mouse 3, respectively.

### Example 3

PCR Amplification, RFLP Analysis, and DNA Sequencing.

Genomic DNA was isolated with DNAzol® (Molecular Research Center, Inc., Cincinnati, Ohio) from the skin biopsy samples and amplified by PCR to generate a 354 bp fragment using the primers, described previously (Alexeev, et al., 1998. Nat Biotech, 16: 1443-1346). The PCR products were digested with DdeI restriction enzyme and reaction products were analyzed by 15% polyacrylamide gel electrophoresis. The PCR products were purified and subjected to automated DNA sequencing using the primer 5′-AAGAATGCTGCCCACCATG-3′ (AB 1 373A; Applied Biosystems, Foster City, Calif.).

Shown in FIG. 5 are RFLP and DNA sequencing analysis of genomic DNA isolated from DOPA-positive skin biopsies and controls. (A) Dde I digestion pattern of wild-type and mutant tyrosinase. The asterisk indicates the position of the point mutation of the tyrosinase gene in albino melanocytes. (B) RFLP analysis from skin biopsies from seven mice treated with Tyr-A and untreated control. (C) DNA sequence analysis of the tyrosinase gene from skin biopsies of BALB/c mice. The arrows designate the targeted base for correction.

### Example 4

Induction of Genetic Skin Disorder by Creating Dominant Mutations in Mice

Shown in FIG. 6 are photographs of RDO treated mice carrying a genotypic change in COL7A1 gene or control mouse. The first three photographs (starting from left) are three BALB/c mice were intradermally injected as described above with 10 μg of MCOL7-A (RDO designed to create a dominant mutation in COL7A1 gene), the fourth photograph is a mouse injected with cytofectin only. Shown in FIG. 7 are photographs of RDO treated mice carrying a genotypic change in KRT17 gene. Four BALB/c mice intradermally injected with 10 μg of MKRT17-A (RDO for KRT17 gene).

All publications and references, including but not limited to patent applications, cited in this specification, are herein incorporated by reference in their entirety as if each individiual publication or reference were specifically and individually indicated to be incorporated by reference herein as being fully set forth.

While this invention has been described with a reference to specific embodiments, it will be obvious to those of ordinary skill in the art that variations in these methods and compositions may be used and that it is intended that the invention may be practiced otherwise than as specifically described herein. Accordingly, this invention includes all modifications encompassed within the spirit and scope of the invention as defined by the claims.

What is claimed is:

1. A method of modifying a selected gene in cells of a human skin at one or more locations which comprises delivering to said cells an effective amount of a composition comprising a chimeric RNA-DNA oligonucleotide and a pharmaceutically acceptable carrier such that the stable genetic modifications are made to the selected gene which result in phenotypic changes at said locations of the human skin wherein the selected gene is naturally expressed in cells of the human skin.

2. The method of claim 1, wherein the stable genetic modification is in an epidermal fragility disorder gene.

3. The method of claim 1, wherein the stable genetic modification is in a keratinization disorder gene.

4. The method of claim 1, wherein the selected gene is tyrosinase, COL7A1, LAMA3, LAMB3, LAMC2, COL17A1, ITGA6, ITGB4, PLEC1, KRT5, KRT14, PKP1, KRT1, KRT10, KRT9, KRT16, LOR, KRT2e, KRT6a, KRT 16, KRT 17, STS, TGM1, GJB2, GJB3, ATP2A2, DSP, DSG1, HR, hHB1, hHB6, PAX3, TYR, TYRP-1, OCA2, OA1, MITF, HPS, FECH, UROS, URO-D, XPA, XPB, XPC, XPD, XPG, CSB, PTC, STK11/LKB1, PTEN, PTEN, XPB, XPD, WHN, GLA, ATM, ENG, ALK-1, or PPO gene.

5. The method of claim 1, wherein the selected gene is tyrosinase gene.

6. The method of claim 1, wherein the selected gene is COL7A1 gene.

7. The method of claim 1, wherein the selected gene is KRT17 gene.

8. The method of claim 1, wherein the chimeric RNA-DNA oligonucleotide comprises:
(a) a first string of nucleotides wherein the first string is made of at least four contiguous deoxyribonucleotides flanked on each side by at least nine ribonucleotides; and
(b) a second string of nucleotides that is fully complementary to the first string of nucleotides or is fully complementary to the first string of nucleotides except that the first and second strings have one mismatched base pair in the region corresponding to the deoxyribonucleotides of the first string, wherein the second string has the same number of deoxyribonucleotides as in the first string of nucleotides, and
wherein one or more nucleotides of the chimeric RNA-DNA oligonucleotide are nuclease protected, and wherein the chimeric RNA-DNA oligonucleotide has nucleotides in the first and second strings that are fully complementary to a segment of DNA of the selected gene except that the first string has one mismatching deoxyribonucleotide that defines the site of modification in the selected gene.

9. The method of claim 1, wherein the chimeric RNA-DNA oligonucleotide comprises:
(a) a first string of nucleotides wherein the first string is made of at least 20 ribonucleotides; and
(b) a second string of deoxyribonucleotides having the same number of deoxyribonucleotides as in the first string of nucleotides, wherein the second string is fully complementary to the first string of nucleotides except that the second string has a deoxyribonucleotide that forms a mismatched base pair with the corresponding nucleotide in the first string, and
wherein one or more nucleotides of the chimeric RNA-DNA oligonucleotide are nuclease protected, and wherein the chimeric RNA-DNA oligonucleotide has nucleotides in the first and second strings that are fully complementary to a segment of the two strands of DNA of the selected gene except that the deoxyribonucleotide in the second string also forms a mismatched base pair with the corresponding deoxyribonucleotide in the DNA strand of the selected gene which mismatched base pair defines the site of modification in the selected gene.

10. The method of claim 1, wherein the chimeric RNA-DNA oligonucleotide comprises:
(a) a first string of nucleotides wherein the first string is made of at least four contiguous deoxyribonucleotides flanked on each side by at least nine ribonucleotides; and
(b) a second string of nucleotides that is fully complementary to the first string of nucleotides or is fully complementary to the first string of nucleotides except that the first and second strings have one mismatched base pair in the region corresponding to the deoxyribonucleotides of the first string, wherein the second string has the same number of deoxyribonucleotides as in the first string of nucleotides, and wherein one or more nucleotides of the chimeric RNA-DNA oligonucleotide are nuclease protected, and wherein the chimeric RNA-DNA oligonucleotide has nucleotides in the first and second strings that are fully complementary to a segment of DNA of the selected gene except that the first and second strings have one, two or four pairs of nucleotide insertions or deletions that defines the site of modification in the selected gene.

11. The method of claim 1, wherein the stable genetic modification is correction of a mutation.

12. The method of claim 11, wherein the mutation is a point mutation or a frame shift mutation.

13. The method of claim 1, wherein the stable genetic modification is generation of a mutation.

14. The method of claim 13, wherein the mutation is a point mutation or a frame shift mutation.

15. The method of claim 13, wherein the mutation is a dominant mutation.

16. The method of claim 1, wherein said phenotypic changes include the correction of a skin disorder.

17. The method of claim 1, wherein said phenotypic changes include the correction of albinism, an epidermal fragility disorder or a keratinization disorder.

18. A method of modifying a selected gene in cells of an animal skin at one or more locations which comprises delivering to said cells an effective amount of a composition comprising a chimeric RNA-DNA oligonucleotide and a pharmaceutically acceptable carrier such that the stable genetic modifications are made to the selected gene which result in phenotypic changes at said locations of the animal skin, wherein the animal is selected from the group consisting of a mouse, a rabbit, a goat, a monkey, a pig and a cow.

19. The method of claim 17, wherein the selected gene is tyrosinase, COL7A1, LAMA3, LAMB3, LAMC2, COL17A1, ITGA6, ITGB4, PLEC1, KRT5, KRT14, PKP1, KRT1, KRT10, KRT9, KRT16, LOR, KRT2, KRT6, KRT 16, KRT 17, STS, TGMI, GJB2, GJB3, ATP2A2, DSP, DSG1, HR, hHB1, hHB6, PAX3, TYR, TYRP-1, OCA2, OA1, MITF, HPS, FECH, UROS, URO-D, PPO, XPA, XPB, XPC, XPD, XPG, CSB, PTC, STK11/LKB 1, PTEN, PTEN, XPB, XPD, WHN, GLA, ATM, ENG, ALK-1, or a cytokine gene.

20. The method of claim 18, wherein the selected gene is tyrosinase gene.

21. The method of claim 18, wherein the selected gene is COL7A1 gene.

22. The method of claim 18, wherein the selected gene is KRT17 gene.

23. The method of claim 18, wherein the chimeric RNA-DNA oligonucleotide comprises:
(a) a first string of nucleotides wherein the first string is made of at least four contiguous deoxyribonucleotides flanked on each side by at least nine ribonucleotides; and
(b) a second string of nucleotides that is fully complementary to the first string of nucleotides or is fully complementary to the first string of nucleotides except that the first and second strings have one mismatched base pair in the region corresponding to the deoxyribonucleotides of the first string, wherein the second string has the same number of deoxyribonucleotides as in the first string of nucleotides, and
wherein one or more nucleotides of the chimeric RNA-DNA oligonucleotide are nuclease protected, and wherein the chimeric RNA-DNA oligonucleotide has nucleotides in the first and second strings that are fully complementary to a segment of DNA of the selected gene except that the first string has one mismatching deoxyribonucleotide that defines the site of modification in the selected gene.

24. The method of claim 18, wherein the chimeric RNA-DNA oligonucleotide comprises:
(a) a first string of nucleotides wherein the first string is made of at least 20 ribonucleotides; and
(b) a second string of deoxyribonucleotides having the same number of deoxyribonucleotides as in the first string of nucleotides, wherein the second string is fully complementary to the first string of nucleotides except that the second string has a deoxyribonucleotide that forms a mismatched base pair with the corresponding nucleotide in the first string to make the genetic modifications in the selected gene, and
wherein one or more nucleotides of the chimeric RNA-DNA oligonucleotide are nuclease protected, and wherein the chimeric RNA-DNA oligonucleotide has nucleotides in the first and second strings that are fully complementary to a segment of the two strands of DNA of the selected gene except that the deoxyribonucleotide in the second string also forms a mismatched base pair with the corresponding deoxyribonucleotide in the DNA strand of the selected gene which mismatched base pair defines the site of modification in the selected gene.

25. The method of claim 18, wherein the chimeric RNA-DNA oligonucleotide comprises:
(a) a first string of nucleotides wherein the first string is made of at least four contiguous deoxyribonucleotides flanked on each side by at least nine ribonucleotides; and
(b) a second string of nucleotides that is fully complementary to the first string of nucleotides or is fully complementary to the first string of nucleotides except that the first and second strings have one mismatched base pair in the region corresponding to the deoxyribonucleotides of the first string, wherein the second string has the same number of deoxyribonucleotides as in the first string of nucleotides, and
wherein one or more nucleotides of the chimeric RNA-DNA oligonucleotide are nuclease protected, and wherein the chimeric RNA-DNA oligonucleotide has nucleotides in the first and second strings that are fully complementary to a segment of DNA of the selected gene except that the first and second strings have one, two or four pairs of nucleotide insertions or deletions that defines the site of modification in the selected gene.

26. The method of claim 18, wherein the stable genetic modification is correction of a mutation.

27. The method of claim 26, wherein the mutation is a point mutation or a frame shift mutation.

28. The method of claim 18, wherein the stable genetic modification is generation of a mutation.

29. The method of claim 28, wherein the mutation is a point mutation or a frame shift mutation.

30. The method of claim 28, wherein the mutation is a dominant mutation.

31. The method of claim 18, wherein said phenotypic changes include the correction of albinism, an epidermal fragility disorder or a keratinization disorder.

32. An animal model having a skin disorder at one or more locations of its skin wherein the skin disorder is a result of a treatment at said locations with a composition comprising a chimeric RNA-DNA oligonucleotide targeted to a selected skin gene, wherein the skin disorder is an epidermal fragility disorder, a keratinization disorder or albinism disorder.

33. The animal model of claim 32, wherein the selected skin gene is Tyr, COL7A1, LAMA3, LAMB3, LAMC2, COL17AI, ITGA6, ITGB4, PLEC1, KRT5, KRT14, PKP1, KRT1, KRT10, KRT9, KRT16, LOR, 1998, KRT2e, KRT6a, KRT 16, KRT 17, STS, TGM1, GJB2, GJB3, ATP2A2, DSP, DSG1, HR, hHB1, hHB6, PAX3, TYR, TYRP-1, OCA2, OA1, MITF, HPS, FECH, UROS, URO-D, PPO, XPA, XPB, XPC, XPD, XPG, CSB, PTC, STK11/LKB1, PTEN, PTEN, XPB, XPD, WHN, GLA, ATM, ENG, ALK-1, or a cytokine gene.

34. The method of claim 33, wherein the selected gene is Tyr gene.

35. The method of claim 33, wherein the selected gene is COL7A1 gene.

36. The method of claim 33, wherein the selected gene is KRT17 gene.

37. The method of claim 32, wherein the skin disorder is due to generation of a mutation in the selected skin gene.

38. The method of claim 37, wherein the mutation is a point mutation or a frame shift mutation.

39. The method of claim 37, wherein the mutation is a dominant mutation.

40. A method of correcting a mutation in a tyrosinase gene in cells of a mammalian skin at one or more locations which comprises delivering to said cells an effective amount of a composition comprising a Tyr-A RNA-DNA oligonucleotide for causing stable genetic correction in the tyrosinase gene and a pharmaceutically acceptable carrier such that the correction results in restoration of tyrosinase enzyme activity at said locations of the mammalian skin, wherein the mammalian skin is selected from the group consisting of a human, a mouse, a rabbit, a goat, a monkey, a pig and a cow.
TABLE 1
Genodermatoses and genes with known gene defects

| Disease | Affected gene | References |
|---|---|---|
| Epidermal | | |
| fragility disorders | | |
| Dystrophic EB | COL7A1 | Uitto, et al., 1996, In: |
| | | Epidermolysis Bullosa: |
| | | Clinical, Epiderniologic |
| | | and Laboratory Advances, |
| | | and the Findings of the |
| | | National Epidermolysis |
| | | Bullosa Registry (Fine J-D, |
| | | Bauer EA, McGuire J, and |
| | | Moshell A, eds.) The Johns |
| | | Hopkins University Press, |
| | | Baltimore, MD, pp. 326-350 |
| Junctional EB | LAMA3, | Pulkkinen et al., 1999, In: |
| | LAMB3, | Epidermolysis Bullosa: |
| | LAMC2 | Clinical, Epiderniologic |
| | | and Laboratory Advances, |
| | | and the Findingi of the |
| | | National Epidermolysis |
| | | Bullosa Registry (Fine, J.-D., |
| | | Bauer, E. A., McGuire, J., |
| | | and Moshell, A., eds.) |
| | | The Johns Hopkins |
| | | University Press, |
| | | Baltimore, MD, pp. 300-325 |
| GABEB | COL17A1 | Pulkkinen et al., 1998, Exp |
| | | Dermatol 7:46 |
| EB-PA | ITGA6, | Pulkkinen et al., 1998, Exp |
| | ITGB4 | Dermatol 7:46 |
| EB-MD | PLEC1 | Uitto et al., 1996, Exp |
| | | Dermatol 5:237 |
| EB-simplex | KRT5, KRT14 | Corden et al., 1996, Exp |
| | | Dermatol 5:297 |
| EDA/skin fragility | PKP1 | McGrath et al., 1997, Nat |
| | | Genet 17:240 |
| Keratinization disorders | | |
| Epidermolytic | KRT1, KRT10 | Corden et al., 1996, Exp |
| hyperkeratosis | | Dermatol 5:297 |
| Epidermolytic | | Corden et al., 1996, Exp |
| PPK KRT9 | | Dermatol 5:297 |
| Non-epidermolytic PPK | KRT16 | Corden et at., 1996, Exp |
| | | Dermatol 5:297 |
| Vohwinkel's syndrome | LOR | Ishida-Yamamoto et al., 1998, |
| | | Exp Dermatol 7:1 |
| Ichthyosis bullosa | KRT2e | Rothnagel JA 1996, Current |
| Siemens | | Op Dermatol 3:127 |
| Pachonychia congenita | KRT6a, 16, 17 | Rothnagel JA 1996, Current |
| type 1/2 | | Op Dermatol 3:127 |
| X-linked ichthyosis | STS | Bonifas et al., 1987, Proc Nat |
| | | Acad Sci 84:9248 |
| Lamellar ichthyosis | TGM1 | Ishida-Yamamoto et al., 1998, |
| | | Exp Dermatol 7:1 |
| Palmoplantar | GJB2 | Richard et al., 1998, Hum |
| keratoderma with | | Genet 103:393 |
| deafness | | |
| Erythrokeratodermia | GJB3 | Richard et al., 1998, Nat |
| variabilis | | Genet 20:366 |
| Darier's disease | ATP2A2 | Sakuntabhai et al., 1999, Nat |
| | | Genet 21:271 |
| Striate palmoplantar | DSP | Armstrong et al., 1999, Hum |
| keratoderma | | Molec Genet 8:143 |
| Striate keratoderma | DSG1 | Rickman et al., 1999, Hum |
| | | Mol Genet, (In Press) |
| Hair disorder | | |
| Congenital atrichia | HR | Ahmad et al., 1998, Science |
| | | 279:720 |
| Monilethrix | hHB1, hHB6 | Korge et al., 1998, J Invest |
| | | Dermatol 111:896; |
| | | Winter et al., 1997, Nat Genet |
| | | 16:372 |
| Pigmentation disorders | | |
| Waardenburg syndrome | PAX3 | Nordlund et al., 1998, Oxford |
| | | Univ Press |
| Albinism | TYR, TYRP-1, | Boissy et al., 1997, Pigment |
| (different forms) | OCA2, OA1 | Cell Res 10:12 |
| Tietz syndrome | MITF | Nordlund et al., 1998, Oxford |
| | | Univ Press |
| Hermansky-Pudlak | HPS | Boissy et al., 1997, Pigment |
| syndrome | | Cell Res 10:12 |
| Porphyrias | | |
| Erythropoietic | FECH | Murphy GM, 1999, Br J |
| protoporphyria | | Dermatol 140:573 |
| Congenital | UROS | Murphy GM, 1999, Br J |
| erythropoietic porphyria | | Dermatol 140:573 |
| Familial porphyria | URO-D | Murphy GM, 1999, Br J |
| cutanen tarda | | Dermatol 140:573 |
| Variegate porphyria | PPO | Murphy GM, 1999, Br J |
| | | Dermatol 140:573 |
| Cancer disorders | | |
| Xeroderma pigmentosum | XPA, XPB, | van Steeg et al., 1999, Mol |
| | XPC, XPD, | Med Today 5:86 |
| | XPG, CSB | |
| Basal cell nevus | PTC | Bale et al, 1998, J Cutan Med |
| syndrome | | Surg 3:31; Ingham PW, 1998, |
| | | Curr Opin Genet Dev 8:88 |
| Peutz-Jeghers | STK11/LKB1 | Dong et al., 1998, Cancer Res |
| | | 58:3787; Rowan et al., 1999, |
| | | J Invest Dermatol 112:509 |
| Cowden syndrome | PTEN | Eng C, 1998, Int J Oncol |
| | | 12:701 |
| Bannayan-Zonan | PTEN | Marsh et al., 1997, Nat Genet |
| syndrome | | 16:333 |
| Multisystem disorders | | |
| Trichothiodystrophy | XPB, XPD | van Steeg et al., 1999, Mol |
| | | Med Today 5:86 |
| Nude | WHN | Frank et al., 1999, Nature |
| | | 398:473 |
| Fabry's disease | GLA | Peters et al., 1997, Postgrad |
| | | Med J 73:710 |
| Ataxia telangiectasia | ATM | Crawford TO, 1998, Sernin |
| | | Pediatr Neurol 5:287 |
| Hereditary hemorrhagic | ENG, ALK-1 | Marchuk DA, 1998, Curr |
| telangiectasia (HHT) | | Opin Hematol 5:332 |

Abbreviations: EB, epidermolysis bullosa; GABEB, generalized atrophic benign EB; PA, pyloric atresia; MD, muscular dystrophy; EDA, ectodermal dysplasia; PPK, palmoplantar keratoderma.
